(19) United States
(12) Patent Application Publication
(10) Pub. No.: US 2025/0084159 A1
(43) Pub. Date: Mar. 13, 2025

(54) MCT11 ANTIBODIES TO TREAT T CELL FUNCTIONAL EXHAUSTION AND ENHANCE CANCER IMMUNOTHERAPY

(71) Applicant: University of Pittsburgh - Of the Commonwealth System of Higher Education, Pittsburgh, PA (US)

(72) Inventors: Greg M. Delgoffe, Pittsburgh, PA (US); Ronal Peralta, Pittsburgh, PA (US)

(73) Assignee: University of Pittsburgh - Of the Commonwealth System of Higher Education, Pittsburgh, PA (US)

(21) Appl. No.: 18/580,741

(22) PCT Filed: Jul. 19, 2022
(86) PCT No.: PCT/US2022/073903

§ 371 Date: Jan. 19, 2024

Related U.S. Application Data
(60) Provisional application No. 63/223,473, filed on Jul. 19, 2021.

(51) Int. Cl. C07K 16/28; A61K 39/00; A61K 47/68; A61P 35/00; A61P 37/06

(52) U.S. Cl. CPC C07K 16/28; A61K 47/6849; A61P 35/00; A61P 37/06; A61K 2039/505; C07K 2317/21; C07K 2317/24; C07K 2317/31; C07K 2317/52; C07K 2317/565

(57) ABSTRACT

Monoclonal antibodies and antigen binding fragments that specifically bind MCT11 are provided. Also disclosed are nucleic acid molecules encoding the antibody, vectors including these nucleic acid molecules, and host cells transfected with these vectors. Methods of using MCT11 specific antibodies to treat cancer, inhibit or treat T cell exhaustion, and/or enhance immunotherapy, are also provided.

### CROSS REFERENCE TO RELATED APPLICATIONS

This claims the benefit of U.S. Provisional Application No. 63/223,473, filed Jul. 19, 2021, which is incorporated by reference herein.

### FIELD

This relates to monoclonal antibodies and antigen binding fragments that specifically bind a monocarboxylate transporter 11 (MCT11), and use thereof for treating cancer, reducing T cell exhaustion, and increasing response to immunotherapy in a subject.

### BACKGROUND

The programmed cell death 1 (PD-1) receptor is a checkpoint receptor mainly expressed on mature cytotoxic T lymphocytes. Cancer cells often express PD-1 ligands, such as PD-L1 and PD-L2, leading to immune tolerance of cancerous cells. Certain cancer therapies target PD-1 or its ligands to reduce immune tolerance, thereby increasing T cell mediated elimination of cancerous cells. However, only a subset of patients respond to this so-called PD-1 blockade. A potential factor limiting efficacy is the development of T cell exhaustion, an alternative differentiation fate of T cells to a dysfunctional state. Exhaustion limits the capacity of T cells to respond to immunotherapy. Thus, therapeutics that increase T cell function may prove an effective strategy to treat cancer, improve cellular therapies (e.g., adoptive cell transfer (ACT) therapy), or improve patient response to various cancer immunotherapies.

### SUMMARY

It is shown here that MCT11 is upregulated in terminally exhausted T cells, especially those that infiltrate tumors. It is also shown here that lactic acid uptake by exhausted T cells is blocked by treatment with an anti-MCT11 antibody. Moreover, treatment with an α-MCT11 mAb is shown to significantly reduce tumor growth in a mouse cancer model. Thus, without being bound to any particular theory, MCT11 mediated uptake of lactic acid (or another MCT11 substrate) may reduce anti-tumor function in T cells. Based on these observations, provided are methods of treating T cell exhaustion, which can enhance cancer treatment, such as T cell-based immunotherapies.

Monoclonal antibodies that specifically bind to monocarboxylate transporter 11 (MCT11) are provided herein. In some embodiments, the antibody or antigen binding fragment includes a heavy chain variable region (V_{H}) and a light chain variable region (V_{L}) including a heavy chain complementarity determining region (CDR)1, a CDR2, and a CDR3, and a light chain complementarity determining region (CDR)1, a CDR2, and a CDR3, of the V_{H} and V_{L} set forth as SEQ ID NOs: 1 and 5, respectively. In some embodiments, the antibody is a monoclonal antibody. In some examples, the monoclonal antibody is an antibody fragment (e.g., antigen binding fragment), for example, an Fab fragment, an Fab′ fragment, an F(ab)′₂ fragment, an Fv fragment, a single chain variable fragment (scFV), a dimer of a single chain antibody (scFV₂), or a disulfide stabilized variable fragment (dsFV). Also disclosed herein are antibody conjugates, for example, the disclosed monoclonal antibody can be linked to an effector molecule or detectable marker. In some embodiments, the antibody conjugate is an antibody-drug conjugate (when the effector molecule is a therapeutic molecule). In further embodiments, the disclosed antibodies are multispecific antibodies that include the disclosed monoclonal antibody specific to MCT11, and at least one additional antibody that binds at least one other antigen, such as PD-1, 4-1BB/CD137, GITR, OX40, CD105, LAG3, TIM-3/HAVCR2, NRP1, or FAS. Also disclosed are nucleic acid molecules encoding the monoclonal antibody disclosed herein, vectors including these nucleic acid molecules, and host cells transformed with these nucleic acids or vectors.

Methods for reducing T cell exhaustion, increasing an effector function of a T cell, or both, are provided. In some embodiments, the T cell is an Adoptive Cell Transfer (ACT) therapy T cell, for example, a tumor-infiltrating lymphocyte (TIL), chimeric antigen receptor T cell (CAR-T), or engineered T cell receptor (TCR) T cell. Also provided are methods for treating cancer (or tumor), treating T cell exhaustion, and/or increasing a response to cancer immunotherapy in a subject. In some examples the subject has cancer and/or is receiving immunotherapy. In some embodiments the disclosed methods increase effector T cell function or reduce T cell exhaustion in the subject.

The foregoing and other objects and features of the disclosure will become more apparent from the following detailed description, which proceeds with reference to the accompanying figures.

### BRIEF DESCRIPTION OF THE DRAWINGS

FIG. 1 shows typical phenotypes of “progenitor-like” or “terminally exhausted” T cells.

FIGS. 2A-2C show that MCT11 is upregulated in mouse and human exhausted T cells. FIG. 2A shows FACS sorting of LN CD8⁺ and TIL CD8⁺ cells for RNA-seq (left), and that MCT11 is upregulated in exhausted T cells (PD1^{hi}Tim3⁺) (right). FIG. 2B shows MCT11 staining of human PBMCs, PD1/TIM3⁻ and PD1/TIM3⁺ cells from melanoma (MEL) or head and neck cancer (HNSCC) patients. Human tumor biopsy samples were stained with antibodies to CD8, PD-1, Tim-3 and MCT11 and analyzed by flow cytometry. FIG. 2B depicts the staining of MCT11 as a function of progression to exhaustion (PD-1+Tim3+). FIG. 2C shows MCT11 surface expression on exhausted or non-exhausted tumor infiltrating lymphocytes (TILs) from MC38 (adenocarcinoma) or MEER (head and neck cancer) models in C56/BL6J mice.

FIGS. 3A-3B show Slc16a11 Transcripts Per Million bases (TPM) from RNA-seq of the indicated cell types. MCT11 is expressed (upregulated) on the surface of exhausted T cells, especially those that infiltrate tumors (TIL) (FIG. 3A). FIG. 3B was generated from publicly available data and confirms that Slc16a11 is specific to tumor-infiltrating exhausted T cells.

FIG. 4 shows that exhausted T cells specifically take up lactic acid. A schematic of the experimental design is shown at the top while the graph below shows lactic acid uptake after a 30 minute incubation with lactic acid for each indicated cell type.

FIG. 5 shows that lactic acid (LA) uptake is specifically blocked when exhausted tumor-infiltrating lymphocytes (TILs) are treated with polyclonal anti-MCT antibodies (labeled as “αMCT11” in this figure).

FIG. 6 shows that lactic acid (LA) uptake is blocked when exhausted tumor-infiltrating lymphocytes (TILs) are treated with monoclonal anti-MCT antibodies (labeled as “MCT11 mAb”).

FIGS. 7A-7C show that α-MCT11 mAb has activity in B16 melanoma and MEER HNSCC models. FIG. 7A shows a diagram of the experimental set-up. FIG. 7B shows tumor growth inhibition in B6 mice bearing B16 melanoma treated with IgG2a, α-PD1, or α-MCT11 mAb (labeled “αMCT11” in this figure). FIG. 7C shows tumor growth inhibition in B6 mice bearing MEER tumors treated with isotype control or α-MCT11 mAb (labeled “anti-MCT11” in this figure).

FIGS. 8A-8C show that the therapeutic effect of α-MCT11 mAb blockade depends at least in part on adaptive immunity. FIG. 8A shows a diagram of the experimental set-up. FIG. 8B shows tumor growth in RAGKO mice bearing B16 melanoma treated with the indicated treatments. FIG. 8C shows tumor growth in RAGKO mice bearing MEER tumors treated with the indicated treatments.

FIGS. 9A-9C show that α-MCT11 mAb functions by blocking, rather than depleting, MCT11-expressing cells. FIG. 9A shows a diagram of the experimental set-up. FIG. 9B shows tumor area following treatment with IgG2a, α-MCT11 mAb (labeled “anti MCT11” in this figure), or an FC mutant of α-MCT11 mAb (labeled “FC mut anti MCT11”). Mice that cleared tumors in response to MCT11 blockade (anti-MCT11 CRs) were inoculated again with MEER tumor cells. FIG. 9C shows that mice that had previously cleared MEER tumors show immunologic memory to the tumor.

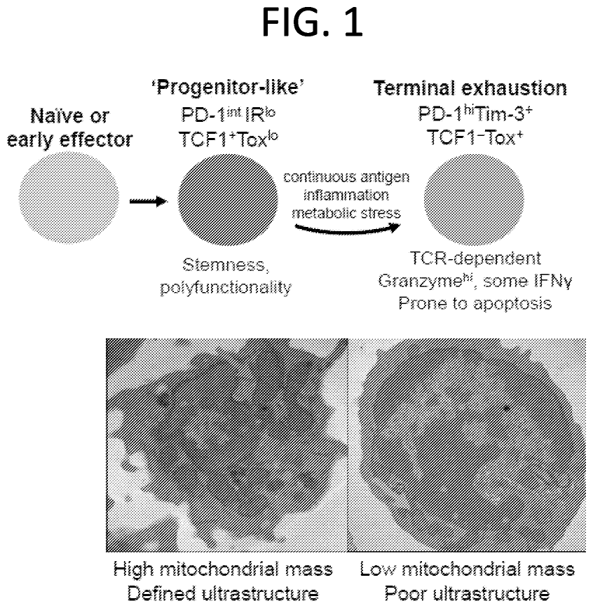

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

The Sequence Listing is submitted as an XML file, “Sequence.xml,” created on Jul. 17, 2022, 20,480 bytes, which is incorporated by reference herein. In the accompanying sequence listing:

SEQ ID NO: 1 is the amino acid sequence of the MCT11 V_{H}.

SEQ ID NOs: 2, 3, and 4 are the amino acid sequences of the CDR1, CDR2, and CDR3, respectively, of the MCT11 V_{H}.

SEQ ID NO: 5 is the amino acid sequence of the MCT11 V_{L}.

SEQ ID NOs: 6, 7, and 8 are the amino acid sequences of the CDR1, CDR2, and CDR3, respectively, of the MCT11 V_{L}.

SEQ ID NO: 9 is an exemplary amino acid sequence of human MCT11.
MPAPQRKHRRGGFSHRCFPTPQTAMTPQPAGPPDGGWGWVVAAAAFAINGLSYGLLRSL
GLAFPDLAEHFDRSAQDTAWISALALAVQQAASPVGSALSTRWGARPVVMVGGVLASLG
FVFSAFASDLLHLYLGLGLLAGFGWALVFAPALGTLSRYFSRRRVLAVGLALTGNGASSLL
LAPALQLLLDTFGWRGALLLLGAITLHLTPCGALLLPLVLPGDPPAPPRSPLAALGLSLFTR
RAFSIFALGTALVGGGYFVPYVHLAPHALDRGLGGYGAALVVAVAAMGDAGARLVCGW
LADQGWVPLPRLLAVFGALTGLGLWVVGLVPVVGGEESWGGPLLAAAVAYGLSAGSYA
PLVFGVLPGLVGVGGVVQATGLVMMLMSLGGLLGPPLSGFLRDETGDFTASFLLSGSLILS
GSFIYIGLPRALPSCGPASPPATPPPETGELLPAPQAVLLSPGGPGSTLDTTC
SEQ ID NO: 10 is an exemplary nucleic acid sequence encoding human SLC16A11
mRNA.
CATTTCTCGCCGCTCCCCCCTCCCCCGGGCCTGGGGGTTGTGTGTGTACATAATTCAAT
CCCCGTGGGACTGGCGTCTGGCCTCACGCGCGGCTTGCCGGATTGGCTGTTTCAGCTCG
CCCCCGCCCTCCGTACACCCCGCGTCCCAGGTGGCTCAGGCCCCTGTGGTGATCTCTGT
TTACCGAGAGAGCCCGTCCAAGTTGGGCTCCATCGCTGCCCTCGCTCCCCTTCGGGGCC
CCCGCCCGCCTGGGAAGCAGAGAGAAAGCCGGGCCCAGCCCTTCCTCACCCTTCCCCT
CCCCGCACCGCCCGGAGAGGTCGGACGGCGATGACCCCCCAGCCCGCCGGACCCCCG
GATGGGGGCTGGGGCTGGGTGGTGGCGGCCGCAGCCTTCGCGATAAACGGGCTGTCCT
ACGGGCTGCTGCGCTCGCTGGGCCTTGCCTTCCCTGACCTTGCCGAGCACTTTGACCGA
AGCGCCCAGGACACTGCGTGGATCAGCGCCCTGGCCCTGGCCGTGCAGCAGGCAGCCA
GCCCCGTGGGCAGCGCCCTGAGCACGCGCTGGGGGGCCCGCCCCGTGGTGATGGTTGG
GGGCGTCCTCGCCTCGCTGGGCTTCGTCTTCTCGGCTTTCGCCAGCGATCTGCTGCATC
TCTACCTCGGCCTGGGCCTCCTCGCTGGCTTTGGTTGGGCCCTGGTGTTCGCCCCCGCC
CTAGGCACCCTCTCGCGTTACTTCTCCCGCCGTCGAGTCTTGGCGGTGGGGCTGGCGCT
CACCGGCAACGGGGCCTCCTCGCTGCTCCTGGCGCCCGCCTTGCAGCTTCTTCTCGATA
CTTTCGGCTGGCGGGGCGCTCTGCTCCTCCTCGGCGCGATCACCCTCCACCTCACCCCC
TGTGGCGCCCTGCTGCTACCCCTGGTCCTTCCTGGAGACCCCCCAGCCCCACCGCGTAG
TCCCCTAGCTGCCCTCGGCCTGAGTCTGTTCACACGCCGGGCCTTCTCAATCTTTGCTCT
AGGCACAGCCCTGGTTGGGGGCGGGTACTTCGTTCCTTACGTGCACTTGGCTCCCCACG
CTTTAGACCGGGGCCTGGGGGGATACGGAGCAGCGCTGGTGGTGGCCGTGGCTGCGAT
GGGGGATGCGGGCGCCCGGCTGGTCTGCGGGTGGCTGGCAGACCAAGGCTGGGTGCC
CCTCCCGCGGCTGCTGGCCGTATTCGGGGCTCTGACTGGGCTGGGGCTGTGGGTGGTG
GGGCTGGTGCCCGTGGTGGGCGGCGAAGAGAGCTGGGGGGGTCCCCTGCTGGCCGCG
GCTGTGGCCTATGGGCTGAGCGCGGGGAGTTACGCCCCGCTGGTTTTCGGTGTACTCCC
CGGGCTGGTGGGCGTCGGAGGTGTGGTGCAGGCCACAGGGCTGGTGATGATGCTGATG
AGCCTCGGGGGGCTCCTGGGCCCTCCCCTGTCAGGCTTCCTAAGGGATGAGACAGGAG
ACTTCACCGCCTCTTTCCTCCTGTCTGGTTCTTTGATCCTCTCCGGCAGCTTCATCTACA
TAGGGTTGCCCAGGGCGCTGCCCTCCTGTGGTCCAGCCTCCCCTCCAGCCACGCCTCCC
CCAGAGACGGGGGAGCTGCTTCCCGCTCCCCAGGCAGTCTTGCTGTCCCCAGGAGGCC
CTGGCTCCACTCTGGACACCACTTGTTGATTATTTTCTTGTTTGAGCCCCTCCCCCAATA
AAGAATTTTTATCGGGTTTTCCTGAAA
SEQ ID NO: 11 is an exemplary immunizing peptide of MCT11.
VHLAPHALDRGLGGYGAALVVAVAAMGDAGARLVCGWLADQGWVPLP
SEQ ID NO: 12 is an exemplary amino acid sequence of mouse MCT11.
MTPKPAGPPDGGWGWVVAAAAFAVNGLSYGLLRSLGLALPDLAEHFERSAQDTAWVSA
LALAVQQAASPVGSALSTRWGARPVVMVGGVLTSLGLVESAFARSLLHLYLGLGLLAGS
GWALVFAPALGTLSRYFSRRRVLAVGLALTGNGASSLLLAPALQFLLDTFGWRGALLLLG
AVTLHLTPCGALLRPLALSGDPLAPPRTPLAALGLGLFKRRAFSVFALGTALIGGGYFVPY
VHLGPHALDQGMGGYGAALVVAVAAVGDACARLASGWLADQGWVPLPRLLVVFGSLT
GLGVLAMGLVPTVGTEEGWGAPLLAAAGAYGLSAGSYAPLVFGVLPGLVGIGGVVQATG
LVMMLMSLGGLLGPPLSGFLRDKTGDFSASFLVCSSFILSGSFIYMGLPRALPSCRPASPPAT
PPPERGELLPVPQVSLLSAGGTGSIRDTTC
SEQ ID NO: 13 is an exemplary nucleic acid sequence encoding mouse Slc16all mRNA.
GAGGCGGGGCGCGGCCACCAGGTACGCTCACTCCTCGCGGTTCCCCAACCAGG
GCTGGGGCTCGTGTGTGGATAATTCAATCCTCGTGGGACTCGGCGTCAGGCCTTAGCC
AGGCCTGGGTGGATTGGCTGTCTCTCTTTCCCCCGCCCTCGGTATTCACCCCCACCCCC
ACCCCGTGTCCCAGGTGGCTTGGGCCCCTGTGGTGATCTCTGTTTACTGGAGAGAGCG
GTCCAAGTTGGGCTCCATCTCTGTGCTGGCTAGCTGCTCCGCAGAACCCTTTCTGCGGA
AAGCGCGGAGAAAATCCGGCCGAACCCACCTTCGCCCTTTCCCTACCCCCAACGCCGG
GAGAGGTCGGCAGACGGCGATGACCCCCAAGCCGGCCGGACCCCCGGACGGGGGCTG
GGGCTGGGTGGTGGCGGCCGCAGCATTCGCCGTGAACGGGCTCTCCTACGGGCTCTTA
CGCTCCCTGGGCCTTGCCCTCCCCGACCTCGCGGAGCACTTTGAACGAAGCGCCCAGG
ACACTGCGTGGGTCAGCGCCCTGGCCTTGGCCGTGCAGCAGGCAGCCAGCCCAGTGGG
CAGCGCCCTGAGCACTCGCTGGGGGGCACGCCCCGTGGTGATGGTTGGGGGAGTCCTA
ACCTCGCTTGGCTTGGTCTTCTCGGCTTTCGCCCGAAGCCTCCTGCACCTCTACCTCGG
CCTGGGCCTCCTCGCTGGCTCTGGCTGGGCCCTGGTGTTTGCCCCAGCCCTGGGTACCC
TCTCTCGGTACTTCTCCCGCCGTCGGGTCTTGGCGGTAGGGTTGGCGCTCACCGGTAAT
GGGGCATCCTCGCTGCTCCTGGCACCTGCCTTGCAGTTCCTCCTTGATACTTTCGGCTG
GCGGGGTGCCTTGCTCCTCCTTGGCGCTGTCACCCTTCACCTCACACCCTGTGGCGCCT
TGCTAAGACCTTTAGCTCTCTCTGGTGACCCGCTGGCCCCACCTCGCACCCCCCTAGCT
GCCCTTGGCCTAGGTCTGTTCAAGCGCCGGGCCTTTTCAGTCTTTGCTTTGGGCACCGC
CTTGATCGGGGGCGGATACTTCGTCCCCTACGTTCATTTGGGTCCGCATGCTTTAGATC
AAGGCATGGGTGGTTATGGGGCAGCGTTAGTGGTGGCTGTCGCTGCAGTGGGAGATGC
CTGTGCCCGATTGGCCAGCGGATGGCTGGCAGACCAGGGCTGGGTGCCCCTTCCGAGG
CTTCTGGTGGTGTTTGGGTCTCTGACTGGGTTAGGGGTACTAGCAATGGGACTAGTGCC
CACTGTGGGGACAGAGGAGGGTTGGGGGGCTCCTCTGCTGGCCGCTGCTGGGGCCTAT
GGGCTGAGCGCTGGGAGTTATGCCCCACTGGTTTTCGGTGTGCTCCCGGGGCTGGTGG
GCATTGGAGGTGTGGTGCAGGCCACAGGGCTGGTGATGATGCTGATGAGCCTCGGGGG
ACTCCTGGGCCCTCCTCTGTCAGGCTTCCTAAGGGATAAGACAGGAGACTTCAGTGCC
TCTTTCCTGGTGTGCAGCTCTTTCATCCTCTCTGGCAGTTTCATCTACATGGGGCTGCCC
AGAGCCCTCCCCTCCTGCCGTCCAGCCTCACCTCCAGCAACCCCTCCACCAGAGAGAG
GGGAGCTGCTCCCAGTTCCACAAGTCTCCCTGCTTTCCGCAGGGGGTACTGGCTCCATC
CGGGATACCACTTGTTGATCATTTTCTTGGTTGACCTCCTTCCCTAATAAAGAATTTTTA
TCTTACAAAAAAAAAAAAAAAAA

### DETAILED DESCRIPTION

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017; The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising an antibody” means “including an antibody” without excluding other elements. It is further to be understood that any and all base sizes given for nucleic acids are approximate, and are provided for descriptive purposes, unless otherwise indicated. Although many methods and materials similar or equivalent to those described herein can be used, particularly suitable methods and materials are described below. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

About: Unless context indicated otherwise, “about” refers to plus or minus 5% of a reference value. For example, “about” 100 refers to 95 to 105.

Administration: To provide or give a subject an agent, such as a MCT11 monoclonal antibody, by any effective route. Administration can be local or systemic. Exemplary routes of administration include, but are not limited to, injection (such as subcutaneous, intramuscular, intradermal, intraperitoneal, intratumoral, intraprostatic, intrathecal, intraarterial, intraosseous, and intravenous), oral, sublingual, rectal, transdermal, intranasal, vaginal and inhalation routes. In some examples, the monoclonal antibody is administered by intravenous injection.

Adoptive Cell Transfer (ACT) Therapy: A type of immunotherapy in which a T cell that has been modified (e.g., modified to recognize a tumor antigen) and/or expanded, is administered to a patient in need thereof. T cells for ACT therapy can be a patient's own T cells (e.g., modified and/or expanded ex vivo), or T cells from a donor. ACT therapies include, for example, Chimeric Antigen Receptor T cell (CAR-T), Engineered T Cell Receptor (TCR), or Tumor-Infiltrating Lymphocyte (TIL) therapies. ACT therapy is also sometimes referred to as adoptive cell therapy, cellular adoptive immunotherapy, or T-cell transfer therapy.

Antibody and Antigen Binding Fragment: An immunoglobulin, antigen-binding fragment, or derivative thereof, that specifically binds and recognizes an analyte (antigen), such as MCT11, such as human MCT11. The term “antibody” is used herein in the broadest sense and encompasses various antibody structures, including but not limited to monoclonal antibodies, polyclonal antibodies, multispecific antibodies (e.g., bispecific antibodies), and antigen binding fragments, so long as they exhibit the desired antigen-binding activity.

Non-limiting examples of antibodies include, for example, intact immunoglobulins and variants and fragments thereof that retain binding affinity for the antigen. Examples of antigen binding fragments include but are not limited to Fv, Fab, Fab′, Fab′-SH, F(ab′)₂; diabodies; linear antibodies; single-chain antibody molecules (e.g., scFv); and multispecific antibodies formed from antibody fragments. Antibody fragments include antigen binding fragments either produced by the modification of whole antibodies or those synthesized de novo using recombinant DNA methodologies (see, e.g., Kontermann and Dübel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In combination, the heavy and the light chain variable regions specifically bind the antigen.

References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, known as “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some embodiments, a disclosed antibody includes a heterologous constant domain. For example, the antibody includes a constant domain that is different from a native constant domain, such as a constant domain including one or more modifications to increase half-life.

A “single-chain antibody” (scFv) is a genetically engineered molecule containing the V_{H} and V_{L} domains of one or more antibody linked by a suitable polypeptide linker as a genetically fused single chain molecule (see, for example, Bird et al., Science, 242:423-426, 1988; Huston et al., Proc. Natl. Acad. Sci., 85:5879-5883, 1988; Ahmad et al., Clin. Dev. Immunol., 2012, doi:10.1155/2012/980250; Marbry, IDrugs, 13:543-549, 2010). The intramolecular orientation of the V_{H}-domain and the V_{L}-domain in a scFv, is typically not decisive for scFvs. Thus, scFvs with both possible arrangements (V_{H}-domain-linker domain-V_{L}-domain; V_{L}-domain-linker domain-V_{H}-domain) may be used. In a dsFv the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994).

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions (see, for example, Greenfield (Ed.), Antibodies: A Laboratory Manual, 2^{nd} ed. New York: Cold Spring Harbor Laboratory Press, 2014.) Monoclonal antibodies include humanized monoclonal antibodies.

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004; Lonberg, Nat. Biotech., 23: 1117-1125, 2005; Lonenberg, Curr. Opin. Immunol., 20:450-459, 2008).

Antibodies also include genetically engineered forms such as chimeric antibodies (such as humanized murine antibodies) and heteroconjugate antibodies (such as bispecific antibodies). See, e.g., Pierce Catalog and Handbook, 1994-1995 (Pierce Chemical Co., Rockford, IL); Kuby, J., Immunology, 3^{rd} Ed., W.H. Freeman & Co., New York, 1997.

Antibody-drug conjugate (ADC): A molecule that includes an antibody (or antigen-binding fragment of an antibody) conjugated to a drug. ADCs can be used to specifically target a drug to particular cells (such as a cancer cell or exhausted T cell) through specific binding of the antibody to a target antigen expressed on the cell surface. Exemplary drugs for use with ADCs include anti-viral agents (e.g., remdesivir, galidesivir, arbidol, favipiravir, baricitinib, or lopinavir/ritonavir), anti-microtubule agents (e.g., maytansinoids, auristatin E and auristatin F), interstrand crosslinking agents (e.g., pyrrolobenzodiazepines; PBDs), calicheamicin family (e.g., ozogamycin), topoisomerase inhibitors (e.g., govitecan/exetecan), PD-1 inhibitors, T cell agonists, or bacterial toxins (e.g., PE38). In some cases, the ADC is a bi-specific ADC, which is comprised of two monoclonal antibodies or antigen-binding fragments thereof, each directed to a different antigen or epitope, conjugated to a drug. In one example, the ADC includes an anti-mammalian MCT11 antibody or antigen binding fragment, such as anti-human MCT11, such as a mAb specific for human MCT11.

Binding affinity: The affinity of an antibody for an antigen. In one embodiment, affinity is calculated by a modification of the Scatchard method described by Frankel et al., Mol. Immunol., 16:101-106, 1979. In another embodiment, binding affinity is measured by an antigen/antibody dissociation rate. In another embodiment, a high binding affinity is measured by a competition radioimmunoassay. In another embodiment, binding affinity is measured by ELISA. In some embodiments, binding affinity is measured using the Octet system (Creative Biolabs), which is based on bio-layer interferometry (BLI) technology. In other embodiments, Kd is measured using surface plasmon resonance assays, e.g., using a BIACORES-2000 or a BIACORES-3000 (BIAcore, Inc., Piscataway, N.J.). In other embodiments, antibody affinity is measured by flow cytometry or by surface plasmon resonance. Other exemplary methods are provided in Harlow & Lane, Antibodies, A Laboratory Manual, 2^{nd} ed., Cold Spring Harbor Publications, New York (2013).

An antibody that “specifically binds” an antigen (such as MCT11, such as human MCT11) is an antibody that binds the antigen with high affinity and does not significantly bind other unrelated antigens. In one example, an antibody that specifically binds MCT11 is an antibody that binds substantially to the MCT11 substrate to which the protein is attached, or the protein in a biological specimen. A certain degree of non-specific interaction may occur between an antibody and a non-target. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking the epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays can be used to select monoclonal antibodies that are specifically immunoreactive with a protein. In some examples, a monoclonal antibody (such as an MCT11 mAb) specifically binds to a target (such as human MCT11) with an equilibrium constant (Kd) of 50 nM or less, such as 45 nM or less, 40 nM or less, 35 nM or less, 30 nM or less, 25 nM or less, 20 nM or less, 15 nM or less, 10 nM or less, or 5 nM or less.

Bispecific antibody: A recombinant protein that includes antigen-binding fragments of two different monoclonal antibodies, and is thereby capable of binding two different antigens or two different epitopes of the same antigen. Similarly, a multispecific antibody is a recombinant protein that includes antigen-binding fragments of at least two different monoclonal antibodies, such as two, three or four different monoclonal antibodies. In one example, the bispecific antibody includes an anti-MCT11 antibody, such as anti-human MCT11, such as a mAb specific for human MCT11.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

Checkpoint Inhibitor (or Checkpoint Blockade): A therapeutic that targets a checkpoint protein. Checkpoints help prevent over-active immune responses or autoimmune responses, and can sometimes limit T cell ability to eliminate cancerous cells. When checkpoints are blocked (e.g., PD-1 blockade) T cells can better target and kill cancerous cells. Examples of checkpoint proteins found on T cells or cancerous cells include PD-1/PD-L1/PD-L2, and CTLA-4/B7-1/B7-2.

Exemplary checkpoint inhibitors include ipilimumab (Yervoy®), nivolumab (Opdivo®), pembrolizumab (Keytruda®), atezolizumab (Tencentriq®), avelumab (Bavencio®), durvalumab (Imfinzi®), cemiplimab (Libtayo®), palbociclib (Ibrance®), ribociclib (Kisquali®), and abemaciclib (Verzenio®). Further examples are provided in Qiu et al., Journal of the European Society for Therapeutic Radiology and Oncology, 126(3):450-464, 2018; Visconti et al., J Exp Clin Cancer Res. 35(1): 153, 2016; and Mills et al. Cancer Res. 77(23): 6489-6498, 2017.

Chimeric antigen receptor (CAR): Artificial, engineered T cell receptors, which graft an arbitrary specificity onto an immune effector cell. Typically, these receptors are used to graft the specificity of a monoclonal antibody onto a T cell (e.g., CAR-T); with transfer of their coding sequence facilitated by vectors. Thus, a CAR that “specifically binds” or is “specific” for an antigen is a CAR that binds the antigen with high affinity and does not significantly bind other unrelated antigens. The present disclosure includes CARs specific for MCT11, for example, a CAR including a MCT11-specific antigen binding fragment (e.g., an scFv specific for MCT11, as disclosed herein).

In ACT therapy, CARs can be useful to treat cancer. For example, T cells (obtained from the patient or from a donor) are modified such that they express receptors specific to the patient's particular cancer. The modified T cells, which can then recognize and kill the cancer cells, are introduced into the patient. First generation CARs typically included the intracellular domain from the CD3 ζ-chain, which is the primary transmitter of signals from endogenous TCRs. Second generation CARs added intracellular signaling domains from various costimulatory protein receptors (e.g., CD28, 41BB, ICOS) to the cytoplasmic tail of the CAR to provide additional signals to the T cell. Third generation CARs combine multiple signaling domains, such as CD3z-CD28-41BB or CD3z-CD28-OX40, to augment potency.

A multispecific CAR is a single CAR molecule comprised of at least two antigen-binding domains (such as scFvs and/or single-domain antibodies) that each bind a different antigen or a different epitope on the same antigen (see, for example, US 2018/0230225). For example, a bispecific CAR refers to a single CAR molecule having two antigen-binding domains that each bind a different antigen. A bicistronic CAR refers to two complete CAR molecules, each containing an antigen-binding moiety that binds a different antigen. In some cases, a bicistronic CAR construct expresses two complete CAR molecules that are linked by a cleavage linker. T cells expressing a bispecific or bicistronic CAR can bind cells that express both of the antigens to which the binding moieties are directed (see, for example, Qin et al., Blood 130:810, 2017; and WO/2018/213337). Any of these CARs can be used with the methods described herein.

Complementarity determining region (CDR): A region of hypervariable amino acid sequence that defines the binding affinity and specificity of an antibody. The light and heavy chains of a mammalian immunoglobulin each have three CDRs, designated light chain CDR1 (sometimes referred to as V_{L}-CDR1 or LCDR1), CDR2 (sometimes referred to as V_{L}-CDR2 or LCDR2), and CDR3 (sometimes referred to as V_{L}-CDR3 or LCDR3); and heavy chain CDR1 (sometimes referred to as V_{H}-CDR1 or HCDR1), CDR2 (sometimes referred to as V_{H}-CDR2 or HCDR2), and CDR3 (sometimes referred to as V_{H}-CDR3 or HCDR3), respectively.

Conditions sufficient to form an immune complex: Conditions which allow an antibody or antigen binding fragment to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See, Greenfield (Ed.), Antibodies: A Laboratory Manual, 2^{nd} ed. New York: Cold Spring Harbor Laboratory Press, 2014, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry (IHC), immunoprecipitation (IP), flow cytometry, immunofluorescence microscopy, ELISA, immunoblotting (for example, Western blot), magnetic resonance imaging (MRI), computed tomography (CT) scans, radiography, and affinity chromatography.

Conjugate: A complex of two molecules linked together, for example, linked together by a covalent bond. In one embodiment, an antibody or antibody fragment (e.g., antigen binding fragment) is linked to an effector molecule or second protein (such as a second antibody). The effector molecule can be, for example, a drug, toxin, therapeutic agent, detectable label, protein, nucleic acid, lipid, nanoparticle, carbohydrate or recombinant virus. An antibody conjugate is often referred to as an “immunoconjugate.” When the conjugate includes an antibody linked to a therapeutic, the conjugate is often referred to as an “antibody-drug conjugate” or “ADC.” Other antibody conjugates include, for example, multispecific (such as bispecific or trispecific) antibodies and chimeric antigen receptors (CARs). In one example, a conjugate includes the monoclonal antibody specific for MCT11 disclosed herein.

A peptide linker (short peptide sequence) can optionally be included between the antibody and the effector molecule or second protein. Because conjugates can be prepared from two molecules with separate functionalities and/or origins, such as an antibody and an effector molecule, they are also sometimes referred to as a “chimera.”

Conservative variants: “Conservative” amino acid substitutions are those substitutions that do not substantially affect or decrease a function of a protein, such as the ability of the protein to interact with a target protein. For example, a MCT11-specific antibody can include up to 1, 2, 3, 4, 5, 6, 7, 8, 9, or up to 10 conservative substitutions compared to a reference antibody sequence and retain specific binding activity for MCT11, for example, human MCT11. The term conservative variation also includes the use of a substituted amino acid in place of an unsubstituted parent amino acid.

Individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the antibody, such as the ability to specifically bind to MCT11. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell, for example, a T cell, by placing an antibody in direct physical association with the cell.

Control: A reference standard. In some embodiments, the control is a negative control. In other embodiments, the control is a positive control. In still other embodiments, the control is a historical control or standard reference value or range of values (such as a previously tested control sample, such as a group of patients with known prognosis or outcome, or group of samples that represent baseline or normal values). In some examples, the control may be a subject not receiving treatment with an agent of interest (e.g., the disclosed monoclonal antibody specific for MCT11) or receiving an alternative treatment, or a baseline reading of the subject prior to treatment with the agent.

A difference between a test sample and a control can be an increase or conversely a decrease. The difference can be a qualitative difference or a quantitative difference, for example a statistically significant difference. In some examples, a difference is an increase relative to a control, for example, an increase of at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%. In other examples, a difference is a decrease relative to a control, for example, a decrease of at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, or at least about 100%.

Degenerate variant: In the context of the present disclosure, a “degenerate variant” refers to a polynucleotide encoding a polypeptide (such as an antibody heavy or light chain) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detectable marker: A detectable molecule (also known as a label) that is conjugated directly or indirectly to a second molecule, such as an antibody (such as an MCT11 antibody), to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Effective amount: The amount of an agent (such as an MCT11 monoclonal antibody disclosed herein) that is sufficient to effect beneficial or desired results. An effective amount (also referred to as a therapeutically effective amount) may vary depending upon one or more of: the subject and disease condition being treated, the weight and age of the subject, the severity of the disease condition, the manner of administration and the like, which can be determined by a clinical practitioner. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In one embodiment, an “effective amount” of a therapeutic agent (e.g., an MCT11 monoclonal antibody disclosed herein) is an amount sufficient to treat cancer (or a tumor) in a subject, for example an amount that decreases the volume/size of a tumor, the weight of a tumor, the number of metastases, reduce the volume/size of a metastasis, the weight of a metastasis, or combinations thereof by at least 10%, at least 20%, at least 25%, at least 50%, at least 70%, at least 75%, at least 80%, at least 90%, at least 95%, at least 100%, at least 200%, at least 300%, at least 400%, at least 500%, or at least 600%. In another embodiment, an “effective amount” is an amount (e.g., of an MCT11 monoclonal antibody disclosed herein) sufficient to increase effector function or activity of a T cell, for example by at least 10%, at least 20%, at least 25%, at least 50%, at least 70%, at least 75%, at least 80%, at least 90%, at least 95%, at least 100%, at least 200%, at least 300%, at least 400%, at least 500%, or at least 600%. In some embodiments, an “effective amount” is an amount (e.g., of an MCT11 monoclonal antibody disclosed herein) sufficient to reduce the activity of a target protein (e.g., MCT11), for example by at least 5%, at least 10%, at least 15%, at least 20%, at least 25%, at least 30%, at least 40%, at least 50%, at least 60%, at least 70%, at least 75%, at least 80%, at least 90%, at least 95%, at least 98%, or even 100%. In further embodiments, an “effective amount” (e.g., of an MCT11 monoclonal antibody disclosed herein) is an amount sufficient to reduce T cell exhaustion, for example by at least 5%, at least 10%, at least 15%, at least 20%, at least 25%, at least 30%, at least 40%, at least 50%, at least 60%, at least 70%, at least 75%, at least 80%, at least 90%, at least 95%, or at least 99%. In some examples, combinations of these effects are achieved. An increase or decrease can be determined relative to a suitable control (e.g., no administration of the therapeutic of interest (e.g., MCT11 antibody) or other suitable control).

Epitope: An antigenic determinant. These are particular chemical groups or peptide sequences on a molecule that are antigenic, meaning that they elicit a specific immune response. An antibody specifically binds a particular antigenic epitope on a polypeptide. In some examples, a disclosed antibody or antigen binding fragment specifically binds a MCT11 epitope. In one example, an epitope is specific for a portion of a contiguous sequence of SEQ ID NO: 9, 11, or 12.

Fc region: The constant region of an antibody excluding the first heavy chain constant domain. Fc region generally refers to the last two heavy chain constant domains of IgA, IgD, and IgG, and the last three heavy chain constant domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region is typically understood to include immunoglobulin domains Cγ2 and Cγ3 and optionally the lower part of the hinge between Cγ1 and Cγ2. Although the boundaries of the Fc region may vary, the human IgG heavy chain Fc region is usually defined to include residues following C226 or P230 to the Fc carboxyl-terminus, wherein the numbering is according to Kabat. For IgA, the Fc region includes immunoglobulin domains Cα2 and Cα3 and optionally the lower part of the hinge between Cα1 and Cα2. In some examples, the antibody specific for MCT11 includes an Fc (e.g., human IgG1 Fc or human IgG4 fc), a fucosylated Fc, or a non-Fcr binding Fc.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a protein, such as an scFv, is expressed in a cell, such as a mammalian cell. Methods for introducing a heterologous nucleic acid molecule in a cell or organism are known, for example methods of transformation, including electroporation, lipofection, particle gun acceleration, or homologous recombination.

Host cell: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. In some examples, the host cell is a human cell, for example, a human T cell (e.g., an exhausted T cell) or PBMC.

IgG: A polypeptide belonging to the class or isotype of antibodies that are substantially encoded by a recognized immunoglobulin gamma gene. In humans, this class comprises IgG₁, IgG₂, IgG₃, and IgG₄.

Immune complex: The binding of an antibody or antigen binding fragment (such as a scFv) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Immunotherapy: A therapy that uses an agent to stimulate or suppress the immune system to treat a disease, such as cancer. Some examples of cancer immunotherapy include immune checkpoint inhibitors, adoptive cell therapy, vaccines, and immune system modulators. An immunotherapy can be an antibody, virus, nucleic acid, protein, Fc-fusion protein, or a cell (e.g., a T cell or NK cell). Specific, non-limiting examples include abemaciclib, atezolizumab, avelumab, axicabtagene ciloleucel, blinotumumabl, cemipilimab, durvalumab, ieramilimab, ipilimumab, nivolumab, palbociclib, pembrolizmab, pidilizumab, relatlimab, ribociclib, urelemab, utolimumab, adoptive cell transfer (ACT) therapy (e.g., chimeric antigen receptor (CAR) (e.g., tisagenlecleucel)), or engineered TCR or tumor-infiltrating lymphocyte (TIL)), and oncolytic viruses (e.g., talimogene laherparepvec (T-VEC)).

Increase or Decrease: A positive or negative change, respectively, in quantity from a control value (such as a value representing no therapeutic agent, such as no MCT11 antibody). An increase is a positive change, such as an increase at least 25%, at least 50%, at least 100%, at least 200%, at least 300%, at least 400% or at least 500%, as compared to the control value. A decrease is a negative change, such as a decrease of at least 10%, at least 20%, at least 30%, at least 40%, at least 50%, at least 60%, at least 70%, at least 75%, at least 80%, at least 85%, at least 90%, at least 95%, at least 98%, at least 99%, or at least 100% decrease as compared to a control value. In some examples, the increase or decrease is statistically significant relative to a suitable control.

For example, in some embodiments, administering an agent (e.g., MCT11 specific antibody disclosed herein) decreases activity of a protein target (e.g., MCT11) by reducing (including eliminating or inhibiting) one or more activities of the target (e.g., lactic acid transport). In some embodiments, activity of MCT11 is reduced at least 10%, at least 25%, at least 50%, at least 75%, at least 90%, at least 95%, at least 99%, at least 99.9%, or even 100% relative to a suitable control (e.g., an amount of MCT11 activity observed in the absence of an agent (e.g., MCT11 antibody)).

In some embodiments, a target cell (e.g., an exhausted T cell) is increased or decreased in a sample or in vivo. For example, in some embodiments contacting a sample with an effective amount of an antibody specific for MCT11, and removing cells bound to the antibody, decreases the number of exhausted T cells (including terminally exhausted T cells) in the sample (e.g., the method depletes the sample of exhausted T cells). In some examples, the method decreases a number of exhausted T cells (such as terminally exhausted T cells) in a PBMC population, for example, by at least 20%, at least 30%, at least 40%, at least 50%, at least 60%, at least 70%, at least 75%, at least 80%, at least 85%, at least 90%, at least 95%, at least 99%, or at least 99.9%. In some embodiments, a target cell (e.g., an exhausted T cell, including terminally exhausted T cells) is reduced in vivo, for example, the antibody is administered to a subject and facilitates elimination of the target cell in vivo, for example, by delivering a cytotoxic agent to the target cell or otherwise inducing cell death. In some examples, the method decreases a number of the target cell (such as exhausted T cells, including terminally exhausted T cells) in vivo, for example, by at least 20%, at least 30%, at least 40%, at least 50%, at least 60%, at least 70%, at least 75%, at least 80%, at least 85%, at least 90%, at least 95%, at least 99%, or at least 99.9%, or more.

Isolated: An “isolated” biological component (such as a monoclonal antibody) has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. For example, T cells isolated from a subject, including a tumor from a subject, or other sample, can be at least 50% pure, such as at least 60%, such as at least 75%, at least 80%, at least 90%, at least 95%, at least 98%, at least 99%, or more, pure.

Linker: A bi-functional molecule that can be used to link two molecules into one contiguous molecule, for example, to link a detectable marker to an antibody. Non-limiting examples of peptide linkers include glycine-serine (GS) linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as the disclosed monoclonal antibody. The linkage can be either by chemical or recombinant means. “Chemical means” refers to a reaction between the antibody moiety and the effector molecule such that there is a covalent bond formed between the two molecules to form one molecule.

Monocarboxylate transporter 11 (MCT11): MCT11 is a recently characterized transporter protein that may transport monocarboxylates, such as lactic acid. MCT11 protein is encoded by Slc16a11 (SLC16A11) gene. Sequences for MCT11/Slc16a11 are publicly available, for example, see GenBank Accession Nos: KJ900348.1, NM_153081.3, NM_153357.3, and NM_001370549.1, which provide exemplary Slc16a11 nucleic acid sequences, and UniProt Accession No. Q8NCK7, GenBank Accession Nos: NP_001357478.1, NP_694721.2 and NP_001099267.2, which provide exemplary MCT11 protein sequences. Examples are also provided in SEQ ID NOS: 9, 10, 12, and 13.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (for example, a promoter that drives expression of the heterologous nucleic acid sequences disclosed herein). Generally, operably linked DNA sequences are contiguous and, where necessary to join two protein coding regions, are in the same reading frame.

Programmed cell death protein 1 (PD-1): A cell surface receptor that belongs to the immunoglobulin superfamily and is expressed on T cells and pro-B cells. PD-1 binds two ligands, PD-L1 and PD-L2. The human form is a 268 amino acid type 1 transmembrane protein. PD-1 is an inhibitory receptor that mediates T-cell exhaustion. PD-1 sequences are publicly available, for example from the GenBank® sequence database (e.g., Accession Nos. NP_005009.2 (mature peptide is aa 21-288), CAA48113.1, NP_001301026.1 (mature peptide is aa 25-288), and CAA48113.1 (mature peptide is aa 21-288) provide exemplary PD-1 protein sequences, while Accession Nos. L27440.1, NM_005018.2, X67914.1, AB898677.1 and EU295528.2 provide exemplary PD-1 nucleic acid sequences).

Pharmaceutically acceptable carriers: The pharmaceutically acceptable carriers useful in this invention are conventional. Remington's Pharmaceutical Sciences, 23rd Edition, Academic Press, Elsevier, (2020), describes compositions and formulations suitable for pharmaceutical delivery of a therapeutic agent, such as an MCT11 monoclonal antibody as disclosed herein.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, 5% human serum albumin, glycerol, or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. Supplementary active compounds can also be incorporated into the compositions.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Examples of promoters include, but are not limited to the SV40 promoter, the CMV enhancer-promoter, the CMV enhancer/β-actin promoter, EF1a, and PGK. Both constitutive and inducible promoters are included (see e.g., Bitter et al., Methods in Enzymology 153:516-544, 1987). Also included are promoter elements sufficient to render promoter-dependent gene expression controllable for cell-type specific, tissue-specific, or inducible by external signals or agents; such elements may be located in the 5′ or 3′ regions of the gene. Promoters produced by recombinant DNA or synthetic techniques can also be used to provide for transcription of the nucleic acid sequences.

Recombinant: A recombinant nucleic acid or amino acid is one that has a sequence that is not naturally occurring, or has a sequence that is made by an artificial combination of two otherwise separated segments of sequence.

Sequence identity: The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences are. Homologs of a polypeptide (or nucleotide sequence) will possess a relatively high degree of sequence identity when aligned using standard methods.

Methods of alignment of sequences for comparison have been described. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Subject: A vertebrate, such as a mammal, for example a human. Mammals include, but are not limited to, murines, simians, humans, farm animals, sport animals, and pets. In some embodiments, the subject is a human. In other embodiments, the subject is a non-human mammal, such as a monkey or other non-human primate, mouse, rat, rabbit, pig, goat, sheep, dog, cat, horse, or cow. In some examples, the subject has cancer (or a tumor), or is receiving a cancer immunotherapy, such as adoptive cell transfer (ACT) therapy. In some examples, the subject is a laboratory animal/organism, such as a mouse, rabbit, or rat.

T cell agonists: an immunotherapy that activates T-cells to promote anti-tumor function. Non-limiting examples include urelumab and utomilumab.

T cells: A white blood cell (lymphocyte) that is an important mediator of the immune response. T cells include, but are not limited to, CD3⁺ T cells, CD4⁺ T cells and CD8⁺ T cells. A CD4⁺ T cell is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In some examples, a CD8⁺ T cell is a cytotoxic T lymphocyte (CTL). CD3+ T cells carry the “cluster of differentiation 3” (CD3) marker, a multimeric protein complex historically known as the T3 complex.

Activated T cells can be detected by an increase in cell proliferation and/or expression of or secretion of one or more cytokines (such as IL-2, IL-4, IL-6, IFN-γ, or TNFα). Activation of CD8⁺ T cells can also be detected by an increase in cytolytic activity in response to an antigen. Exhausted T cells are dysfunctional T cells (hyporesponsive) commonly found in cancer environments. T cell exhaustion is characterized by a progressive loss of effector function (for example, loss of IL-2, TNF-α, and IFN-γ production) and sustained expression of inhibitory receptors such as PD-1, T cell immunoglobulin domain and mucin domain-containing protein 3 (Tim-3), CTLA-4, lymphocyte-activation gene 3 (LAG-3), and CD160. In some examples, the exhausted T cell is a CD3⁺ T cell or CD8⁺ T cell. In some examples, the exhausted T cell is a terminally exhausted T cell (a terminally differentiated T cell that is exhausted). Terminally exhausted T cells express Tim3 and have high and persistent expression of PD-1 relative to other T cells (Tim3⁺ PD-1^{hi} T cells). T cells that are Tim3⁺ and/or PD-1^{hi} can be determined by FACs analysis, for example, by FACs analysis of a population of T cells. In some examples, terminally exhausted T cells express MCT11. A possible cause of T cell exhaustion is chronic activation or prolonged antigen stimulation.

T cell receptor (TCR): A receptor found on the surface of T lymphocytes (or T cells) responsible for recognizing fragments of antigen as peptides bound to major histocompatibility complex (MHC) molecules. The TCR is composed of two different protein chains. In humans, in 95% of T cells the TCR consists of an alpha (α) and beta (β) chain, whereas in 5% of T cells the TCR consists of gamma and delta (γ/δ) chains. This ratio changes during ontogeny and in diseased states as well as in different species. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors. In one example, a TCR is a recombinant TCR, such as one used in TCR-engineered T cells for ACT therapy.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Exemplary methods of transformation include chemical methods (e.g., calcium-phosphate transfection), physical methods (e.g., electroporation, microinjection, particle bombardment), fusion (e.g., liposomes), lipofection, nucleofection, receptor-mediated endocytosis (e.g., DNA-protein complexes, viral envelope/capsid-DNA complexes), particle gun accelerator (gene gun), and by biological infection by viruses such as recombinant viruses (Wolff, J. A., ed, Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, blood and other clinical tests, and the like. In some examples, treatment with the disclosed methods results in a decrease in the number, volume, and/or weight of a tumor and/or metastases. In some examples, treatment with the disclosed methods results in a reduction in T cell exhaustion, such as a reduction in the number of exhausted T cells (e.g., Tim3⁺ PD-1^{hi} T cells) in a subject. In some examples, treatment with the disclosed methods reduces T cell exhaustion, such as reducing lactic acid uptake by a T cell, such as an exhausted T cell (e.g., Tim3⁺ PD-1^{hi} T cell). In some examples, combinations of these affects are achieved.

Tumor-Infiltrating Lymphocyte (TIL): Lymphocytes that invade tumor tissue. For example, T cells found with a tumor sample. In ACT therapy, TIL therapy generally involves isolating TILs from a patient tumor, activating and expanding the TILs in culture, and then re-infusing into the patient. In some examples, a T cell is a TIL.

Tumor, neoplasia, or malignancy: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Exemplary tumors, such as cancers, that can be treated using the disclosed MCT11 monoclonal antibodies include solid tumors, such as breast carcinomas (e.g. lobular and duct carcinomas, such as a triple negative breast cancer), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma, and lymphatic tumors (including B-cell and T-cell malignant lymphoma). In one example, the tumor is a melanoma. In one example, the tumor is a head and neck squamous cell carcinoma (HNSCC), such as an HPV-positive HNSCC.

The disclosed MCT11 monoclonal antibodies can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

Vector: A nucleic acid molecule that can be introduced into a host cell (for example, by transfection or transduction), thereby producing a transformed host cell (such as a transformed exhausted T cell). Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors (such as AVV) are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function.

### II. Description of Several Embodiments

Monoclonal antibodies that specifically bind monocarboxylate transporter 11 (MCT11) are provided herein. In some examples, the antibody specifically binds human MCT11. In some embodiments, the monoclonal antibody is an antibody fragment (e.g., antigen binding fragment). Also disclosed are multispecific antibodies (e.g., bispecific antibodies) including the monoclonal antibody or antibody fragment (e.g., antigen binding fragment) that specifically binds MCT11. The multispecific antibodies recognize at least one antigen in addition to MCT11. The antibodies are useful, for example, for treating cancer, reducing T cell exhaustion or increasing effector function of a T cell, or increasing a response to cancer immunotherapy of a subject.

### A. Monoclonal Antibodies and Fragments Thereof That Specifically Bind MCT11

Disclosed herein are isolated monoclonal antibodies that specifically bind MCT11, for example, a mammalian MCT11, such as SEQ ID NO: 9 or 12. In some examples, the monoclonal antibodies specifically bind human MCT11 or a portion thereof (such as SEQ ID NO: 9 or 11). The disclosed antibody includes a V_{H} and a V_{L} domain including a CDR1, CDR2, and CDR3, respectively. In some embodiments, the antibody includes a V_{H} domain including the heavy chain CDR1, CDR2, and CDR3 of SEQ ID NO: 1 and/or a V_{L} domain including the light chain CDR1, CDR2, and CDR3 of SEQ ID NO: 5. Various CDR numbering schemes (such as the Kabat, Chothia or IMGT numbering schemes) can be used to determine CDR positions. In some examples, the numbering scheme used to determine the CDRs is the Chothia numbering scheme (see, e.g., Table 1). In a non-limiting example, the monoclonal antibody includes the amino acid sequences and CDRs of the heavy and light chain as provided in Table 1.

TABLE 1
Chothia CDRs of MCT11-specific antibody and SEQ ID NOs
V_{H}
QVTLKESGPGILQPSQTLSLTCSFSGFSLSTSGLGVGWIRQPSGKGLEWLSHIWWND
VKRYNPALESRLTISKDTSSSQVFLKIASVDTSDTATYYCARIIRSSLITGAVDYWGQ
GTSVTVSSAKTTAPSVYPLAPVCGDTTGSSVTLGCLVKGYFPEPVTLTWNSGSLSSG
VHTFPAVLQSDLYTLSSSVTVTSSTWPSQSITCNVAHPASSTKVDKKIEPRGPTIKPCP
PCKCPAPNLLGGPSVFIFPPKIKDVLMISLSPIVTCVVVDVSEDDPDVQISWFVNNVE
VHTAQTQTHREDYNSTLRVVSALPIQHQDWMSGKEFKCKVNNKDLPAPIERTISKP
KGSVRAPQVYVLPPPEEEMTKKQVTLTCMVTDFMPEDIYVEWTNNGKTELNYKNT
EPVLDSDGSYFMYSKLRVEKKNWVERNSYSCSVVHEGLHNHHTTKSFSRTPG (SEQ
ID NO: 1)

| | SEQ ID NO: 1 | | CDR |
|---|---|---|---|
| V_{H} | positions | CDR protein sequence | SEQ ID NO |
| CDR1 | 26-34 | GFSLSTSGL | 2 |
| CDR2 | 54-58 | WWNDV | 3 |
| CDR3 | 100-112 | IIRSSLITGAVDY | 4 |

V_{L}
DIQMTQTTSSLSASLGDRVTISCSASQGIYNYLNWYRQKPDGTVELLIYYTSSLHSG
VPSRFSGSGSGTAYSLTISNLEPEDFATYYCQQYRKLPWTFGGGTKLEIKRADAAPT
VSIFPPSSEQLTSGGASVVCFLNNFYPKDINVKWKIDGSERQNGVLNSWTDQDSKDS
TYSMSSTLTLTKDEYERHNSYTCEATHKTSTSPIVKSFNRNEC (SEQ ID NO: 5)

| | SEQ ID NO: 5 | | CDR |
|---|---|---|---|
| VL | positions | CDR protein sequence | SEQ ID NO |
| CDR1 | 24-34 | SASQGIYNYLN | 6 |
| CDR2 | 50-56 | YTSSLHS | 7 |
| CDR3 | 89-97 | QQYRKLPWT | 8 |

In some embodiments, the antibody includes a V_{H} including an amino acid sequence at least 90% (such as at least 95%, at least 96%, at least 97%, at least 98%, or at least 99%) identical to the amino acid sequence set forth as SEQ ID NO: 1. In some examples, the antibody includes a V_{L} including an amino acid sequence at least 90% (such as at least 95%, at least 96%, at least 97%, at least 98%, or at least 99%) identical to the amino acid sequence set forth as SEQ ID NO: 5. In additional examples, the antibody includes a V_{H} and a V_{L} independently include amino acid sequences at least 90% (such as at least 95%, at least 96%, at least 97%, at least 98%, or at least 99%) identical to the amino acid sequences set forth as SEQ ID NOs: 1 and 5, respectively.

In some embodiments, the antibody includes a V_{H} including an amino acid sequences at least 90% (such as at least 95%, at least 96%, at least 97%, at least 98%, or at least 99%) identical to the amino acid sequences set forth as SEQ ID NO: 1 and includes the heavy chain CDR1, CDR2, and CDR3 of SEQ ID NOs: 2, 3, and 4, respectively, and also includes a V_{L} including an amino acid sequences at least 90% (such as at least 95%, at least 96%, at least 97%, at least 98%, or at least 99%) identical to the amino acid sequences set forth as SEQ ID NO: 5 and includes the light chain CDR1, CDR2, and CDR3 of SEQ ID NOs: 6, 7, and 8, respectively. Thus, in this particular embodiment, variations due to sequence identity fall outside the CDRs.

In some examples, the antibody includes a V_{H} including SEQ ID NO: 1 and/or includes a V_{L} including SEQ ID NO: 5. In some embodiments, the antibody includes a V_{H} consisting of SEQ ID NO: 1 and/or includes a V_{L} consisting of SEQ ID NO: 5.

In some embodiments, the monoclonal antibody is an antigen binding fragment. Antigen binding fragments are antibody fragments that retain the ability to selectively bind an antigen (e.g., an MCT 11 antigen, such as human MCT11). Non-limiting examples of such fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the V_{H} and V_{L} expressed as two chains; and
(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.
(6) A dimer of a single chain antibody (scFV₂), defined as a dimer of a scFV. This has also been termed a “mini-antibody.”
(7) a disulfide stabilized variable fragment (dsFv)

In some embodiments, the monoclonal antibody is an antigen binding fragment selected from a group consisting of: an Fab fragment, an Fab′ fragment, an F(ab)′₂ fragment, an Fv, a single chain variable fragment (scFV), a dimer of a single chain antibody (scFV₂), and a disulfide stabilized variable fragment (dsFV). In some examples, the antigen binding fragment is an scFv that specifically binds MCT11.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. Other suitable methods of preparing antigen binding fragments have been described, for example, in Harlow and Lane, Antibodies: A Laboratory Manual, 2^{nd}, Cold Spring Harbor Laboratory, New York, 2013.

In some embodiments, the antibody is a non-human mammal or bird antibody, for example, mouse, rat, guinea pig, hamster, rabbit, chicken, dog, cat, sheep, pig, goat, or horse. In specific, non-limiting examples, the antibody is a mouse antibody. In some examples, the antibody is a humanized. In some examples, the antibody is a human antibody. In further examples, the antibody is a chimera, for example, an antibody with variable regions from one species (e.g., mouse) and constant regions from another species (e.g., human). In some embodiments, the antibody includes a constant region. The constant region may include at least one modification to increase half-life, stability and/or function of the monoclonal antibody.

The antibody can include any suitable framework region, such as (but not limited to) a human framework region, or an optimized or humanized framework region. Alternatively, a heterologous framework region, such as, but not limited to a mouse or monkey framework region, can be included in the heavy or light chain of the antibodies.

The antibody can be of any isotype. The antibody can be, for example, an IgA, IgM or an IgG antibody, such as IgG₁, IgG₂, IgG₃, or IgG₄. The class of an antibody that specifically binds to MCT11 can be switched to another, for example, an antibody that specifically binds MCT11 that was originally IgG may be class switched to an IgA. A class can be switched, for example, by changing the constant-region portion of the antibody to that of another class, but keeping the variable region the same. Class switching can be used to convert one IgG subclass to another, such as from IgG₁ to IgG₂, IgG₃, or IgG₄.

The antibody can be derivatized or linked to another molecule (such as another peptide or protein). In some examples, the antibody is linked (by chemical coupling, genetic fusion, noncovalent association or otherwise) to one or more other molecules, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag). In general, the antibody or antigen binding fragment is derivatized such that the binding to MCT11 is not affected adversely by the derivatization or labeling.

In some examples, the disclosed antibodies are oligomers, such as dimers, trimers, tetramers, pentamers, hexamers, septamers, octomers and so on.

In some embodiments, the antibody specifically binds MCT11 with an affinity (e.g., measured by K_{D} (the equilibrium dissociation constant between the antibody and its antigen)) of no more than 1.0×10⁻⁸ M, no more than 5.0×10⁻⁸ M, no more than 1.0×10⁻⁹ M, no more than 5.0×10⁻⁹ M, no more than 1.0×10⁻¹⁰ M, no more than 5.0×10⁻¹⁰ M, no more than 1.0×10⁻¹¹ M, or no more than 5.0×10⁻¹¹ M. In some examples, the K_{D} value is about 10⁻⁴-10⁻⁶ M, 10⁻⁷-10⁻⁹ M, 10⁻¹⁰-10⁻¹² M, 10⁻¹³-10⁻¹⁵ M, 10⁻⁶-10⁻⁹ M, 10⁻⁵-10⁻⁶ M, 10⁻⁵-10⁻⁷ M, or 10⁻⁵-10⁻⁹ M. The K_{D} can be measured, for example, by a radiolabeled antigen binding assay (RIA) performed with the Fab version of an antibody of interest and its antigen. Other methods include ELISA-based methods, micro-scale thermophoresis (MST), surface plasmon resonance (SPR), and biolayer interferometry (BLI). In some examples, the K_{D} is measured using SPR, for example, on a BIACORE®-2000 or a BIACORE®-3000 (BIAcore, Inc., Piscataway, N.J.) see, e.g., Chen et al., J. Mol. Biol. 293:865-881 (1999). In some examples, the K_{D} is cross-validated using two or more methods, for example, by determining K_{D} by both SPR and BLI methods.

### I. Multispecific Antibodies

Also disclosed are multispecific antibodies (e.g., bispecific antibody), that includes the antibody that specifically binds MCT11 as disclosed herein. In some examples, the multispecific antibody includes at least one other antibody that targets an antigen that is not an MCT11 antigen, for example, an antibody that targets PD-1, 4-1BB/CD137, GITR, OX40, CD105, LAG3, TIM-3/HAVCR2, NRP1, or FAS. In some examples, the multispecific antibody includes at least one other antibody that targets a T cell antigen, for example, to target the multispecific antibody to a T cell. In further examples, the multispecific antibody includes the MCT11-specific monoclonal antibody disclosed herein, and at least one other antibody that targets a different antigen of MCT11.

Any suitable method can be used to design and produce the multispecific antibody, such as crosslinking two or more antibodies (e.g., crosslinking the monoclonal antibody disclosed herein and an antibody that binds another antigen) or antigen binding fragments (such as scFvs) of the same type or of different types. Exemplary methods of making multispecific antibodies include those described in PCT Pub. No. WO2013/163427. Non-limiting examples of suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). The multispecific antibody may have any suitable format that allows for binding to MCT11 by the antibody as provided herein.

Bispecific single chain antibodies can be encoded by a single nucleic acid molecule. Non-limiting examples of bispecific single chain antibodies, as well as methods of constructing such antibodies are provided in U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538. Additional examples of bispecific single chain antibodies can be found in PCT application No. WO 99/54440; Mack et al., J. Immunol., 158(8):3965-3970, 1997; Mack et al., Proc. Natl. Acad. Sci. U.S.A., 92(15):7021-7025, 1995; Kufer et al., Cancer Immunol. Immunother., 45(3-4):193-197, 1997; Löffler et al., Blood, 95(6):2098-2103, 2000; and Brühl et al., J. Immunol., 166(4):2420-2426, 2001. Production of bispecific Fab-scFv (“bibody”) molecules are described, for example, in Schoonjans et al. (J. Immunol., 165(12):7050-7057, 2000) and Willems et al. (J. Chromatogr. B Analyt. Technol. Biomed Life Sci. 786(1-2):161-176, 2003). For bibodies, a scFv molecule can be fused to one of the VL-CL (L) or VH-CH1 chains, e.g., to produce a bibody one scFv is fused to the C-term of a Fab chain.

The outermost or N-terminal variable domain is termed VD1 and the innermost variable domain is termed VD2; the VD2 is proximal to the C-terminal CH1 or CL. DVD-immunoglobulin molecules can be manufactured and purified to homogeneity in large quantities, have pharmacological properties similar to those of a conventional IgG₁, and show in vivo efficacy. Any of the disclosed monoclonal antibodies can be included in a DVD-immunoglobulin format.

### II. Antibody Conjugates

The antibodies, antigen binding fragments, or multispecific antibodies (e.g., bispecific antibodies) disclosed herein can be conjugated to an agent, such as an effector molecule or detectable marker. An effector molecule or detectable marker can be covalently or noncovalently attached to the disclosed antibodies, antigen binding fragments, or multispecific antibodies. Various effector molecules and detectable markers can be used, the choice of a particular effector molecule or detectable marker depends on the particular target molecule or cell, and the desired biological effect.

In some examples, the effector molecule is a receptor or receptor fragment (including artificial receptors). In a specific, non-limiting example, the antibody conjugate is a chimeric antigen receptor (CAR) including an MCT11-specific antigen binding fragment (e.g., an scFv specific for MCT11, as disclosed herein).

In some examples, the effector molecule is a drug (e.g., antibody-drug conjugate). Exemplary drugs include anti-viral agents (e.g., remdesivir, galidesivir, arbidol, favipiravir, baricitinib, or lopinavir/ritonavir), anti-microtubule agents (e.g., maytansinoids, auristatin E and auristatin F), interstrand crosslinking agents (e.g., pyrrolobenzodiazepines; PBDs), calicheamicin family (e.g., ozogamycin), topoisomerase inhibitors (e.g., govitecan/exetecan), PD-1 inhibitors, T cell agonists, or bacterial toxins (e.g., PE38). In some cases, the ADC is a bispecific ADC, which is comprised of two monoclonal antibodies or antigen-fragments thereof, each directed to a different antigen or epitope, conjugated to a drug.

A detectable marker is a marker capable of detection, for example, by ELISA, spectrophotometry, flow cytometry, microscopy or diagnostic imaging techniques (such as CT, computed axial tomography (CAT), MRI, magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody, antigen binding fragment, or multispecific antibody, can also be conjugated with enzymes that are useful for detection, such as horseradish peroxidase, β-galactosidase, luciferase, alkaline phosphatase, glucose oxidase and the like. When an antibody is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody, antigen binding fragment, or multispecific antibody, may also be conjugated with biotin, and detected through indirect measurement of avidin or streptavidin binding. It should be noted that the avidin itself can be conjugated with an enzyme or a fluorescent label.

The antibody, antigen binding fragment or multispecific antibody, can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody, antigen binding fragment, or multispecific antibody, may also be labeled with a predetermined polypeptide epitope recognized by a secondary reporter (such as leucine zipper pair sequences, binding sites for secondary antibodies, metal binding domains, epitope tags).

The antibody, antigen binding fragment or multispecific antibody, can also be conjugated with a radiolabeled amino acid, for example, for diagnostic purposes. For instance, the radiolabel may be used to detect exhausted T cells by radiography, emission spectra, or other diagnostic techniques. Examples of labels for polypeptides include, but are not limited to, the following radioisotopes: ³H, ¹⁴C, ³⁵S, ⁹⁰Y, ^{99m}Tc, ¹¹¹In, ¹²⁵I, ¹³¹I. The radiolabels may be detected, for example, using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of detectable marker moieties per antibody, antigen binding fragment, or multispecific antibody in a conjugate can range, for example, from 1 to 20 moieties per antibody or antigen binding fragment. In some embodiments, the average number of effector molecules or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule per antibody ratio) of a conjugate may be controlled in different ways, for example, by: (i) limiting the molar excess of effector molecule-linker intermediate or linker reagent relative to antibody, (ii) limiting the conjugation reaction time or temperature, (iii) partial or limiting reducing conditions for cysteine thiol modification, (iv) engineering by recombinant techniques the amino acid sequence of the antibody such that the number and position of cysteine residues is modified for control of the number or position of linker-effector molecule attachments.

The procedure for attaching an effector molecule or detectable marker to an antibody, antigen binding fragment, or multispecific antibody varies according to the chemical structure of the effector or detectable marker. Polypeptides typically contain a variety of functional groups, such as carboxyl (—COOH), free amine (—NH₂) or sulfhydryl (—SH) groups, which are available for reaction with a suitable functional group on a polypeptide to result in the binding of the effector molecule or detectable marker. Alternatively, the antibody, antigen binding fragment, or multispecific antibody, is derivatized to expose or attach additional reactive functional groups. The derivatization may involve attachment of any suitable linker molecule. The linker is capable of forming covalent bonds to both the antibody or antigen binding fragment and to the effector molecule or detectable marker. Suitable linkers include, but are not limited to, straight or branched-chain carbon linkers, heterocyclic carbon linkers, or peptide linkers. Where the antibody, antigen binding fragment, or multispecific antibody, and the effector molecule or detectable marker are polypeptides, the linkers may be joined to the constituent amino acids through their side chains (such as through a disulfide linkage to cysteine) or the alpha carbon, or through the amino, and/or carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to an antibody, antigen binding fragment, or multispecific antibody (e.g., bispecific antibody) can be determined.

### III. Variants

In some embodiments, amino acid sequence variants of the antibodies, antigen binding fragments, and multispecific antibodies (e.g., bispecific antibodies) disclosed herein are provided. For example, it may be desirable to improve the binding affinity and/or other biological properties. Amino acid sequence variants may be prepared by introducing appropriate modifications into the nucleotide sequence encoding the antibody V_{H} domain and/or V_{L} domain, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics (e.g., specific binding of MCT11, such as human MCT11).

In some embodiments, variants having one or more amino acid substitutions are provided. Sites of interest for substitutional mutagenesis include the CDRs and the framework regions. Amino acid substitutions may be introduced into an antibody of interest and the products screened for a desired activity (e.g., retained/improved antigen binding, decreased MCT11 activity, or improved T cell specificity). Variants typically retain amino acid residues necessary for correct folding and stabilizing between the V_{H} and the V_{L} regions, and will retain the charge characteristics of the residues in order to preserve the low pI and low toxicity of the molecules. Amino acid substitutions can be made in the V_{H} and the V_{L} regions to increase antibody yield of a host cell.

In some embodiments, the V_{H} of the antibody includes up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody includes up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 5. In some examples, the amino acid substitutions are not within the CDR sequences (outside the CDR regions). In such embodiments, the variant antibody retains specific binding of MCT11.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some examples, a substitution, insertion, or deletion in a CDR increases binding affinity for MCT11. In some embodiments of the variant V_{H} and V_{L} sequences provided above, each CDR contains no more than one, two, or three amino acid substitutions. In some examples, the CDRs contain no substitutions. In some embodiments of the variant V_{H} and V_{L} sequences, only the framework residues are modified so the CDRs are unchanged.

To increase binding affinity of the antibody, the V_{L} and V_{H} segments can be randomly mutated, such as within heavy chain CDR3 region or the light chain CDR3 region, in a process analogous to the in vivo somatic mutation process responsible for affinity maturation of antibodies during a natural immune response. Thus in vitro affinity maturation can be accomplished by amplifying V_{H} and V_{L} regions using PCR primers complementary to the heavy chain CDR3 or light chain CDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for MCT11. In particular examples, the V_{H} amino acid sequence is SEQ ID NO: 1. In other examples, the V_{L} amino acid sequence is SEQ ID NO: 5.

In some embodiments, an antibody, antigen binding fragment, or multispecific antibody is altered to increase or decrease the extent to which the antibody or antigen binding fragment is glycosylated. Addition or deletion of glycosylation sites may be accomplished by altering the amino acid sequence such that one or more glycosylation sites is created or removed. Where the antibody includes an Fc region, the carbohydrate attached thereto may be altered. Native antibodies produced by mammalian cells typically include a branched, biantennary oligosaccharide that is generally attached by an N-linkage to Asn297 of the CH₂ domain of the Fc region. In some examples, the antibody (e.g., an antibody specific for MCT11) includes an Fc (e.g., human IgG1 Fc or human IgG4 fc), an afucosylated Fc, or a non-FcR binding Fc. See, e.g., Wright et al. Trends Biotechnol. 15(1):26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

Antibody variants are further provided with bisected oligosaccharides, e.g., a biantennary oligosaccharide attached to the Fc region of the antibody is bisected by GlcNAc. Such antibody variants may have reduced fucosylation. In some examples, the antibody specific for MCT11 includes an afucosylated Fc. Antibody variants with at least one galactose residue in the oligosaccharide attached to the Fc region are also provided.

In several embodiments, the constant region of the disclosed antibodies include one or more amino acid substitutions to optimize in vivo half-life of the antibody. The serum half-life of IgG Abs is regulated by the neonatal Fc receptor (FcRn). Thus, in several embodiments, the antibody includes an amino acid substitution that increases binding to the FcRn. Non-limiting examples of such substitutions include substitutions at IgG constant regions T250Q and M428L (see, e.g., Hinton et al., J Immunol., 176(1):346-356, 2006); M428L and N434S (the “LS” mutation, see, e.g., Zalevsky, et al., Nature Biotechnol., 28(2):157-159, 2010); N434A (see, e.g., Petkova et al., Int. Immunol., 18(12):1759-1769, 2006); T307A, E380A, and N434A (see, e.g., Petkova et al., Int. Immunol., 18(12):1759-1769, 2006); and M252Y, S254T, and T256E (see, e.g., Dall'Acqua et al., J. Biol. Chem., 281(33):23514-23524, 2006). The disclosed antibodies and antigen binding fragments can be linked to or include an Fc polypeptide including any of the substitutions listed above, for example, the Fc polypeptide can include the M428L and N434S substitutions.

In some embodiments, the antibodies disclosed herein may be further modified to contain additional non-proteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

### IV. Additional Description

Antibodies and antigen binding fragments that specifically bind to the same epitope (e.g., the same MCT11 epitope that the disclosed monoclonal antibody binds) can be identified and isolated, for example, by screening combinatorial libraries for antibodies with the desired binding characteristics. In some examples, a phage display library is generated and screened for antibodies possessing desired binding characteristics (e.g., binding to MCT11). Such methods are described, for example, in Hoogenboom et al. in Methods in Molecular Biology 178:1-37 (O'Brien et al., ed., Human Press, Totowa, N.J., 2001); McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of V_{H} and V_{L} genes are separately cloned by polymerase chain reaction (PCR) and recombined randomly in phage libraries, which can then be screened for antigen-binding phage, for example, as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Competitive binding assays, similar to those disclosed in the examples section below, can be used to select antibodies with the desired binding characteristics.

In some examples, antibodies that bind to an epitope of interest (e.g., a MCT11 epitope) can be identified based on their ability to cross-compete (for example, to competitively inhibit the binding of, in a statistically significant manner) with the antibodies provided herein in binding assays.

Human antibodies that bind to the same epitope of MCT11 (e.g., the same MCT11 epitope that the disclosed monoclonal antibody binds) can be produced using any suitable method. Such antibodies may be prepared, for example, by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Additional human antibodies that bind to the same epitope can also be made by hybridoma-based methods. Human myeloma and mouse-human heteromyeloma cell lines for the production of human monoclonal antibodies have been described (see, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005). Human antibodies may also be generated by isolating Fv clone variable domain sequences selected from human-derived phage display libraries. Such variable domain sequences may then be combined with a desired human constant domain.

### B. Polynucleotides and Expression

Nucleic acid molecules (for example, DNA, cDNA, or RNA (e.g., mRNA)) encoding the amino acid sequences of antibodies, antigen binding fragments, multispecific antibodies (e.g., bispecific antibodies), or conjugates that specifically bind MCT11, as described herein, are also provided. Nucleic acids encoding these molecules can readily be produced using the amino acid sequences provided herein (such as the V_{H} and V_{L} sequences and respective CDR sequences listed in Table 1), sequences available in the art (such as framework or constant region sequences), and the genetic code. The genetic code can be used to construct a variety of functionally equivalent nucleic acid sequences, such as nucleic acids that differ in sequence, but encode the same antibody sequence, or encode a conjugate or fusion protein including the V_{L} and/or V_{H} nucleic acid sequence. In some embodiments, the nucleic acid molecules encode the heavy chain CDR1, CDR2, and CDR3 of SEQ ID NOs: 2, 3, and 4, respectively, and encode the light chain CDR1, CDR2, and CDR3 of SEQ ID NOs: 6, 7, and 8, respectively. In several embodiments, the nucleic acid molecules encode the V_{H}, the V_{L}, or both the V_{H} and V_{L} of SEQ ID NOs: 1 and 5, respectively.

Degenerate variants of the disclosed nucleic acid sequences are also disclosed. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA.

The disclosed nucleic acid sequences can be codon optimized for expression in a particular host cell, such as human, mouse, or bacteria. Codon preferences and codon usage tables for a particular species can be used to engineer isolated nucleic acid molecules encoding protein products, such as the disclosed monoclonal antibody, that take advantage of the codon usage preferences of that particular species. For example, the nucleic acid can be designed to have codons that are preferentially used by a particular organism of interest (e.g., the organism to express the monoclonal antibody). In some examples, the nucleic acids are codon optimized for expression in human. In some examples, the nucleic acids are codon optimized for a particular protein expression system, such as bacteria, yeast, insect, or CHO expression systems.

The disclosed nucleic acids can be prepared by any suitable method including, for example, cloning of appropriate sequences or by direct chemical synthesis by standard methods. In some examples, the disclosed nucleic acids are prepared by cloning techniques. Examples of suitable cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements). The nucleic acids can also be prepared by amplification methods. Amplification methods include the polymerase chain reaction (PCR), the ligase chain reaction (LCR), the transcription-based amplification system (TAS), the self-sustained sequence replication system (3SR), and the Qβ replicase amplification system (QB). A wide variety of cloning and in vitro amplification methodologies have been previously described. In some examples, the disclosed nucleic acids are prepared by direct chemical synthesis. Many methods of chemical synthesis have been described, for example, the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is generally limited to sequences of about 100 bases, longer sequences may be obtained by the ligation of shorter sequences.

In some embodiments, the disclosed nucleic acids are included in an expression vector (e.g., viral vector, plasmid, or other vehicle) for expression in a host cell (e.g., human cell, T cell, exhausted T cell, protein expression cell (e.g., bacteria, insect, yeast, Chinese hamster ovary (CHO), human embryonic kidney (HEK293)). In some examples, the vector includes a selectable marker (such as an antibiotic resistance gene (e.g., puromycin) or a reporter gene (e.g., green fluorescent protein (GFP)). In other examples, a selectable marker and/or reporter is not included in the vector. In some examples, the expression vector includes a promoter operably linked to a disclosed nucleic acid molecule. For example, a promoter can be operably linked to a nucleic acid encoding the monoclonal antibody, antigen binding fragment, multispecific antibody, or antibody conjugate specific to MCT11, as disclosed herein. The promoter can be constitutive or inducible. The promoter can be any promoter of interest, including a cytomegalovirus promoter. Typical expression vectors can include sequences useful for regulation of the expression of the DNA encoding the protein (e.g., the disclosed MCT11 specific antibody), for example, appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

In some embodiments, the disclosed nucleic acids are included in a viral vector. Exemplary viral vectors that can be used include, but are not limited to, polyoma, SV40, adenovirus, vaccinia virus, adeno-associated virus (AAV), herpes viruses including HSV and EBV, Sindbis viruses, alphaviruses and retroviruses of avian, murine, and human origin. Baculovirus (Autographa californica multinuclear polyhedrosis virus; AcMNPV) vectors can also be used. Other suitable vectors include orthopox vectors, avipox vectors, fowlpox vectors, capripox vectors, suipox vectors, lentiviral vectors, alpha virus vectors, and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus and adenoviruses, and RNA viruses such as retroviruses and polio. Biologically functional viral and plasmid DNA vectors capable of expression and replication in a cell (e.g., T cell, exhausted T cell, bacteria, yeast, insect, CHO, HEK293, human) are known and a suitable vector can be identified by one of ordinary skill in the art.

The disclosed nucleic acids or vectors encoding the disclosed nucleic acids can be expressed in vitro in a host cell, or in vivo in a cell of a host organism, by DNA transfer into the cell. In some examples, the nucleic acids or vectors disclosed herein are expressed in vitro. The host cell may be prokaryotic or eukaryotic. Numerous expression systems are available for the expression of proteins, including E. coli, other bacterial hosts, yeast, insect, or various eukaryotic cells such as COS, Chinese hamster ovary (CHO), HeLa, myeloma cell, human embryonic kidney (HEK293) lines. Any of these cell lines can be used to express the antibodies, antigen binding fragments, multispecific antibodies, or conjugates disclosed herein. In some embodiments, the host cell is a T cell or an exhausted T cell. Methods of stable transfer, meaning that the foreign DNA is continuously maintained in the host, may be used.

To obtain optimal expression of the disclosed nucleic acids, expression cassettes or vectors can contain, for example, a strong promoter to direct transcription, a ribosome binding site for translational initiation (e.g., internal ribosomal binding sequences), and a transcription/translation terminator can be used. For expression in bacterial cells, such as E. coli, a promoter, such as the T7, trp, lac, or lamda promoters, a ribosome binding site, and preferably a transcription termination signal can be used. For eukaryotic cells, such as human, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). Additional operational elements include, but are not limited to, leader sequence, termination codons, polyadenylation signals and any other sequences necessary for the appropriate transcription and subsequent translation of the nucleic acid sequence.

The disclosed nucleic acids or vectors can be introduced into the host cell by any suitable method (e.g., transformation). Numerous methods of transformation are known, such as: chemical methods (e.g., calcium-phosphate transfection), physical methods (e.g., electroporation, microinjection, particle bombardment), fusion (e.g., liposomes), lipofection, nucleofection, receptor-mediated endocytosis (e.g., DNA-protein complexes, viral envelope/capsid-DNA complexes), particle gun accelerator (gene gun), or by biological infection by viruses such as recombinant viruses (Wolff, J. A., ed, Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA. Successfully transformed cells can be selected by resistance to antibiotics conferred by genes contained in the vector, such as the amp, gpt, neo and hyg genes.

Modifications can be made to the disclosed nucleic acids without diminishing biological activity of the encoded product. For example, modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

The antibodies, antigen binding fragments, multispecific antibodies, and conjugates can be expressed as individual proteins including the V_{H} and/or V_{L} (linked to an effector molecule or detectable marker as needed), or can be expressed as a fusion protein. An immunoadhesin can also be expressed. Thus, in some examples, nucleic acids encoding a V_{H} and V_{L} (e.g., a V_{H} including the CDR sequences of SEQ ID NOs: 2, 3, and 4, and a V_{L} including the CDR sequences of SEQ ID NOs: 6, 7, and 8) and immunoadhesin are provided. The nucleic acid sequences can optionally encode a leader sequence.

To create an scFv, the V_{H}- and V_{L}-encoding DNA fragments can be operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker (see, e.g., Bird et al., Science, 242(4877):423-426, 1988; Huston et al., Proc. Natl. Acad. Sci. U.S.A., 85(16):5879-5883, 1988; McCafferty et al., Nature, 348:552-554, 1990; Kontermann and Dübel (Eds.), Antibody Engineering, Vols. 1-2, 2^{nd} ed., Springer-Verlag, 2010; Greenfield (Ed.), Antibodies: A Laboratory Manual, 2^{nd} ed. New York: Cold Spring Harbor Laboratory Press, 2014). Optionally, a cleavage site can be included in a linker, such as a furin cleavage site. The scFv may be monovalent, for example, if only a single V_{H} and V_{L} are used, bivalent, if two V_{H} and V_{L} are used, or polyvalent, if more than two V_{H} and V_{L} are used. Multispecific or polyvalent antibodies may be generated that bind specifically to MCT11, and another antigen. The encoded V_{H} and V_{L} optionally can include a furin cleavage site between the V_{H} and V_{L} domains. Linkers can also be encoded, such as when the nucleic acid molecule encodes a bi-specific antibody in DVD-Ig™ format.

Any suitable method of expressing and isolating antibodies and antigen binding fragments may be used; including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). Additional non-limiting examples are provided in Al-Rubeai (Ed.), Antibody Expression and Production, Dordrecht; New York: Springer, 2011). Isolated antibodies, antigen binding fragment, multispecific antibodies, or conjugates need not be 100% pure. In some examples, the isolated antibody is substantially free of harmful contaminants (e.g., compositions that are harmful if administered to a human).

Methods for expression of antibodies, antigen binding fragments, multispecific antibodies, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### C. Compositions and Dosages

Compositions are provided that include one or more of the disclosed monoclonal antibodies, antigen binding fragments, multispecific antibodies, conjugates, nucleic acid molecules or vector encoding such molecules, in a pharmaceutically acceptable carrier. In some embodiments, the composition includes a monoclonal antibody specific for MCT11, or an antigen binding fragment thereof. The compositions are useful, for example, for treating cancer (or a tumor), increasing effector function of T cells, increasing resistance to T cell exhaustion (or conversely decreasing T cell exhaustion), increasing response of a subject to immunotherapy, or combinations of these effects. In some examples, the composition is useful for increasing response of a subject to immunotherapy, for example, increasing response to a checkpoint inhibitor, oncolytic virus (e.g., T-VEC), or adoptive cell transfer (ACT) therapy. In some examples, the checkpoint inhibitor is an inhibitor targeting PD-1, CTLA-4, CDK4, CDK6, and/or ligands thereof. In specific, non-limiting examples, the checkpoint inhibitor is ipilimumab, nivolumab, pembrolizumab, atezolizumab, avelumab, durvalumab, cemiplimab, palbociclib, ribociclib, and abemaciclib.

The compositions can be prepared in unit dosage forms, such as in a kit, for administration to a subject. The amount and timing of administration are at the discretion of the administering physician to achieve the desired purposes. The composition can be formulated for systemic or local administration. In one example, the, antigen binding fragment, multispecific antibody, conjugate, or nucleic acid molecule encoding such molecules, is formulated for parenteral administration, such as intravenous administration.

In some embodiments, the composition is at least 70% (such as at least 75%, at least 80%, at least 85%, at least 90%, at least 95%, at least 96%, at least 97%, at least 98%, at least 99%, at least 99.9%, or at least 99.99%) pure. In some embodiments, the composition contains less than 10% (such as less than 5%, less than 4%, less than 3%, less than 2%, less than 1%, less than 0.5%, or even less) of contaminants, such as other proteins or macromolecules (e.g., human, bacterial, yeast).

The composition can be a solution of the MCT11 antibody, antigen binding fragment, multispecific antibody (e.g., bispecific antibody), conjugate, or nucleic acid molecule or vector encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by any suitable technique. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

Any suitable method may be used for preparing administrable compositions; non-limiting examples are provided in such publications as Remington: The Science and Practice of Pharmacy, 22^{nd} ed., London, UK: Pharmaceutical Press, 2013. In some embodiments, the composition is a liquid formulation and includes one or more of the disclosed MCT11 antibodies, antigen binding fragments, multispecific antibodies, or conjugates in a concentration of about 0.1 mg/ml to about 50 mg/ml, for example, about 0.5 mg/ml to about 50 mg/ml, or from about 1 mg/ml to about 50 mg/ml, about 5 mg/ml to about 50 mg/ml, about 10 mg/ml to about 50 mg/ml, about 15 mg/ml to about 50 mg/ml, about 20 mg/ml to 50 mg/ml, about 25 mg/ml to 50 mg/ml, about 30 mg/ml to 50 mg/ml, about 35 mg/ml to 50 mg/ml, about 40 mg/ml to 50 mg/ml, about 45 mg/ml to 50 mg/ml, about 0.1 mg/ml to about 25 mg/ml, from about 0.5 mg/ml to about 25 mg/ml, from about 1 mg/ml to about 25 mg/ml, about 5 mg/ml to about 25 mg/ml, about 0.1 mg/ml to about 15 mg/ml, from about 0.5 mg/ml to about 15 mg/ml, from about 1 mg/ml to about 15 mg/ml, about 5 mg/ml to about 15 mg/ml, about 10 mg/ml to about 25 mg/ml, about 15 mg/ml to about 25 mg/ml, about 20 mg/ml to about 25 mg/ml, about 10 mg/ml to about 40 mg/ml, about 10 mg/ml to about 30 mg/ml, or about 15 mg/ml to about 30 mg/ml. In a specific non-limiting example, the composition includes about 10 to 25 mg/ml or about 50 to 200 mg/ml of the disclosed MCT11 monoclonal antibody, antigen binding fragment, multispecific antibody, or conjugate disclosed herein.

In some embodiments, the composition includes one or more of the disclosed MCT11 antibodies, antigen binding fragments, multispecific antibodies, or conjugates at a concentration of about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 25 mg/ml, 30 mg/ml, 35 mg/ml, 40 mg/ml, 45 mg/ml, or 50 mg/ml. In some examples, the composition includes at least 1 mg, at least 10 mg, at least 25 mg, at least 50 mg, at least 100 mg, at least 200 mg or at least 500 mg, such as 1 mg to 1 g, such as 1 mg to 500 mg, or 10 to 100 mg, of one or more of the disclosed MCT11 antibodies, antigen binding fragments, multispecific antibodies, or conjugates.

The composition containing disclosed MCT11 antibodies, antigen binding fragments thereof, multispecific antibodies, conjugates, nucleic acids or vectors encoding such molecules, can be provided in lyophilized form and rehydrated with a suitable sterile solution before administration. The solution can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997. The composition can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 μm are generally referred to as nanoparticles, nanospheres, and nanocapsules, respectively, and can be administered intravenously. Microparticles are typically around 100 μm in diameter and are administered subcutaneously or intramuscularly. See, for example, Kreuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the compositions disclosed herein. Any suitable polymer may be used, such as a degradable or nondegradable polymeric matrix designed for use in controlled drug delivery. Alternatively, hydroxyapatite has been used as a microcarrier for controlled release of proteins. In yet another aspect, liposomes are used for controlled release as well as drug targeting of the lipid-capsulated drug.

### D. Methods of Use

Disclosed herein are methods of using MCT11 antibodies, such as one or more of those provided herein, to treat T cell exhaustion or increase effector function of a T cell. For example, a therapeutically effective amount of one or more MCT11 antibodies, such as one or more of those provided herein, can be administered to a subject, such as a subject with cancer, such as a cancer that can be treated with an immunotherapy (e.g., ACT therapy). In some examples, a single dose of an MCT11 antibody (or fragment or conjugate thereof) is administered. In some examples, multiple doses of an MCT11 antibody (or fragment or conjugate thereof) is administered, such as at least two, at least three, at least four, at least five, or more separate doses. In some examples, a nucleic acid molecule, such as a vector, encoding the MCT11 antibody (or fragment or conjugate thereof) is administered (e.g., in one or more separate doses, such as at least two, at least three, at least four, at least five, or more separate doses).

In some examples, the MCT11 antibody used in the disclosed methods is one provided herein (e.g., the monoclonal MCT11 antibody or antigen binding fragment specific for MCT11). In a non-limiting example, the MCT11 antibody used in the disclosed methods specifically binds monocarboxylate transporter 11 (MCT11) and includes the variable heavy (V_{H}) domain of SEQ ID NO: 1 and the variable light (V_{L}) domain of SEQ ID NO: 5, respectively. In a further example, the MCT11 antibody used in the disclosed methods specifically binds monocarboxylate transporter 11 (MCT11) and includes an amino acid sequence having at least 90% identity to SEQ ID NO: 1, and includes the variable heavy (V_{H}) CDR1, CDR2, and CDR3 of SEQ ID NOS: 2, 3, and 4, respectively; and includes an amino acid sequence having at least 90% identity to SEQ ID NO: 5, and includes the variable light (V_{L}) CDR1, CDR2, and CDR3 of SEQ ID NOS: 6, 7, and 8, respectively.

In some examples, the MCT11 antibody used in the disclosed methods is a commercially available MCT11 antibody, such as an MCT11 antagonistic antibody or antigen binding fragment specific for MCT11 (e.g., Anti-MCT11 Antibody (G-4) sc-515145 from Santa Cruz Biotechnology; Anti-SLC16A11 from USBiological Life Science (item no: 041802-APC.200); Anti-MCT11 from MyBioSource (Cat #: MBS8292652); MCT-MCT11 from Abcam (ab230845); Anti-MCT1/Monocarboxylic acid transporter 1 antibody (ERR13706(B)) from Abcam (Ab179832); SLC16A11 Polyclonal Antibody from Thermo (Cat #: PA5-98710); SLC16A11 antibody from Biorbyt (Cat. #: Orb376095)). In some examples, the MCT11 antibody is part of an antibody-drug conjugate (ADC), or a bi-specific antibody that specifically binds MCT11 and another antigen. The other antigen can be a T-cell specific antigen. Examples of other antigens include, but are not limited to: PD-1, 4-1BB/CD137, GITR, OX40, CD105, LAG3, TIM-3/HAVCR2, NRP1, or FAS.

In some examples the methods include contacting a T cell with an effective amount of the monoclonal antibody disclosed herein, or by expressing a nucleic acid molecule or vector encoding the monoclonal antibody disclosed herein in a T cell, thereby reducing T cell exhaustion or increasing effector function of a T cell. In some examples, contacting includes administering to a subject with an exhausted T cell or decreased T cell effector function. In some examples, the subject has cancer, or has or will receive an immunotherapy (e.g., abemaciclib, atezolizumab, avelumab, axicabtagene ciloleucel, blinotumumabl, cemipilimab, durvalumab, ieramilimab, ipilimumab, nivolumab, palbociclib, pembrolizmab, pidilizumab, relatlimab, ribociclib, urelemab, utolimumab, adoptive cell transfer (ACT) therapy (e.g., chimeric antigen receptor (CAR) (e.g., tisagenlecleucel)), or engineered TCR or tumor-infiltrating lymphocyte (TIL)), and oncolytic viruses (e.g., talimogene laherparepvec (T-VEC)).

In some examples, the T cell is isolated from a subject (e.g., a donor subject, or the subject with the exhausted T cell or decreased T cell effector function) prior to the contacting step. In some examples, the T cell is a peripheral blood mononuclear cell (PBMC). In some examples, the T cell is a tumor infiltrating lymphocyte (TIL). In some examples, the T cell is reactive to a tumor-specific antigen, for example, one or more of CD19, CD20, BCMA, MUC1, PSA, CEA, HER1, HER2, TRP-2, EpCAM, GPC3, mesothelin 1 (MSLN), or EGFR. In some examples, the T cell is an exhausted T cell. In some examples, the T cell is a terminally exhausted T cell. Cells (e.g., PBMCs, T cells, exhausted T cells) can be isolated from the subject, for example, from a blood sample (e.g., a venous blood sample), biopsy (e.g., tumor sample), or other sample, from the subject. Several techniques for isolating cells of interest are known, for example, density centrifugation (the Ficoll approach), isolation by cell preparation tubes (CPTs), or isolation by SepMate™ tubes. In some examples, apheresis or leukapheresis is used. Flow cytometry techniques (e.g., FACS) can be used to assess the composition of a population of cells (e.g., PBMCs isolated from a subject), for example, to identify cell types, such as monocytes (e.g., CD14), T cells (e.g., CD3, CD8, CD4), B cells (e.g., CD20), or NK cells (e.g., CD56). FACS techniques can also be used to enrich or deplete a particular cell type from a population of cells (for example, enrich or deplete cells positive for CD14, CD3, CD8, CD4, CD28, CD20, CD56, TIM3, PD-1, or combinations thereof). In some examples, T cells are isolated from a PBMC sample, or the PBMC sample is enriched for T cells, for example, isolated or enriched for CD3⁺ or CD8⁺ T cells. In some examples, a sample is enriched by negative selection, for example, by selecting and removing unwanted cell types from a sample (e.g., cell types other than T cells, naïve or memory T cells, exhausted T cells). In some examples, FACS is used to assess whether exhausted T cells (e.g., PD-1^{hi}, TIM3⁺ T cells) are present in a sample, to sort a sample to enrich for exhausted T cells, or conversely to remove exhausted T cells.

In a specific, non-limiting example, TILs are first isolated from a tumor sample (e.g., biopsy) from a subject having cancer or a tumor, and are subsequently contacted with an effective amount of a MCT11-specific antibody, or the TILs are modified to express a nucleic acid molecule or vector encoding a MCT11-specific antibody, for example, one or more of the MCT11-specific antibodies disclosed herein, thereby increasing effector function of the TILs. In such examples, the effective amount can be, for example, an amount that improves in vitro expansion of the TILs (e.g., TILs that are specific to a tumor antigen of a cancer (or tumor) in the subject), and/or improves effector activity of the TILs (e.g., increased cytokine production, increased cytotoxic activity, or decreased expression of T cell exhaustion markers such as TIM3 or PD-1).

Also disclosed herein are methods of treating cancer or a tumor, increasing a response to immunotherapy, or increasing an immune response in a subject by administering an effective amount of an MCT11 monoclonal antibody disclosed herein; or administering an effective amount of the nucleic acid or vector encoding an MCT11 monoclonal antibody disclosed herein, thereby treating the cancer or tumor, or increasing the response to immunotherapy in a subject. In specific, non-limiting examples, the method is a method of treating cancer or a tumor in a subject.

In some examples, the effective amount is an amount sufficient to prevent, treat, reduce, and/or ameliorate one or more signs or symptoms of cancer (or a tumor) in the subject. For example, an amount sufficient to reduce tumor size or tumor load in the subject by at least 10%, at least 20%, at least 30%, at least 40%, at least 50%, at least 60%, at least 70%, at least 80%, at least 90%, at least 95%, or 100%, as compared to a baseline measurement for the same subject, or a suitable control. In some examples, the effective amount is an amount sufficient to inhibit or slow metastasis in the subject. For example, by decreasing tumor spread in the subject by at least 10%, at least 20%, at least 30%, at least 40%, at least 50%, at least 60%, at least 70%, at least 80%, at least 90%, at least 95%, or 100% as compared to a baseline measurement for the same subject, or a suitable control. In some examples, the effective amount is an amount that increases life expectancy of the subject, for example, by at least 10%, at least 20%, at least 30%, at least 40%, at least 50%, at least 60%, at least 70%, at least 80%, at least 90%, at least 100%, at least 200%, at least 300%, at least 400%, or more. In other examples, the effective amount is an amount sufficient to reduce tumor density in the subject, for example, by at least 10%, at least 20%, at least 30%, at least 40%, at least 50%, at least 60%, at least 70%, at least 80%, at least 90%, or 100% as compared to a baseline measurement for the same subject or other suitable control. Non-limiting examples of suitable controls include untreated subjects or subjects not receiving the monoclonal antibody or nucleic acid or vector encoding the monoclonal antibody disclosed herein (e.g., subjects receiving other agents or alternative therapies). In further examples, the effective amount is an amount sufficient to target and eliminate tumor cells, for example, eliminate at least 10%, at least 20%, at least 30%, at least 40%, at least 50%, at least 60%, at least 70%, at least 80%, at least 85%, at least 90%, at least 95%, at least 98%, or even 100%, relative to a suitable control.

In some embodiments, an effective amount of the MCT11 monoclonal antibody is an amount that blocks transport of monocarboxylates by at least one cell expressing MCT11 (e.g., an exhausted T cell, a regulatory T cell, or a resident memory T cell). In some embodiments, the MCT11 monoclonal antibody blocks lactic acid, pyruvic acid, ketone bodies, butyrate, proprionate, or succinate uptake by at least one cell expressing MCT11 (e.g., an exhausted T cell, a regulatory T cell, or a resident memory T cell).

In some embodiments, the subject is receiving, has received, or will receive at least one immunotherapy (e.g., abemaciclib, atezolizumab, avelumab, axicabtagene ciloleucel, blinotumumabl, cemipilimab, durvalumab, ieramilimab, ipilimumab, nivolumab, palbociclib, pembrolizmab, pidilizumab, relatlimab, ribociclib, urelemab, utolimumab, adoptive cell transfer (ACT) therapy (e.g., chimeric antigen receptor (CAR) (e.g., tisagenlecleucel)), or engineered TCR or tumor-infiltrating lymphocyte (TIL)), and oncolytic viruses (e.g., talimogene laherparepvec (T-VEC)). In some examples, the immunotherapy includes an adoptive cell transfer (ACT) therapy, for example, a CAR (e.g., CAR-T or CAR-NK), TCR, or TIL immunotherapy. In specific, non-limiting examples, the immunotherapy is a CAR-T therapy. In some examples, the immunotherapy includes a checkpoint inhibitor, for example, a checkpoint inhibitor targeting PD-1, PD-L1, CD137, CD223, CTLA-4, CDK4, and/or CDK6. Exemplary checkpoint inhibitors include ipilimumab, urelemab, ieramilimab, nivolumab, pembrolizumab, atezolizumab, avelumab, durvalumab, cemiplimab, palbociclib, ribociclib, and abemaciclib. In some embodiments, administering an immunotherapy increases an immune response in the subject, for example, in a subject having cancer.

In some embodiments, increasing a response to immunotherapy includes preventing, treating, reducing, and/or ameliorating one or more signs or symptoms of cancer in the subject. In some embodiments, the effective amount of the monoclonal antibody or nucleic acid or vector encoding the monoclonal antibody disclosed herein, is an amount that when administered with an immunotherapy, the combination is more effective at treating cancer (or a tumor) relative to administration of the immunotherapy alone; for example, in some examples, the combination is at least 10%, at least 20%, at least 30%, at least 40%, at least 50%, at least 60%, at least 70%, at least 80%, at least 90%, at least 100%, at least 200%, at least 300%, at least 400%, at least 500%, or more effective (preventing, treating, reducing, and/or ameliorating one or more sign or symptom of cancer or a tumor) than treatment of the immunotherapy alone. In some examples, the effective amount is an amount that is synergistic when administered with the immunotherapy, for example, an amount that synergistically prevents, treats, reduces, and/or ameliorates one or more sign or symptom of cancer.

In some embodiments, the subject has cancer. In some examples, the subject has a solid tumor or cancer, such as breast carcinomas (e.g. lobular and duct carcinomas, such as a triple negative breast cancer), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma (HNSCC), and lymphatic tumors (including B-cell and T-cell malignant lymphoma).

In some examples, the subject has a liquid tumor or cancer, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

In a specific, non-limiting example, the subject has leukemia, colorectal cancer, cervical cancer, lung cancer, bladder cancer, head and neck cancer, pancreatic cancer, glioblastoma, head and neck squamous cell carcinoma, ovarian cancer, uterine cancer, prostate cancer, breast cancer, melanoma, non-small cell lung cancer (NSCLC), renal cell carcinoma, sarcomas, or adrenal carcinoma. In another specific, non-limiting example, the subject has melanoma or head and neck squamous cell carcinoma (HNSCC).

In some embodiments, the methods disclosed herein reduce T cell exhaustion. In some examples, the methods reduce T cell exhaustion in a subject (e.g., a subject having cancer or receiving an immunotherapy). In some examples, T cell exhaustion is decreased by at least 10%, at least 20%, at least 30%, at least 40%, at least 50%, at least 60%, at least 70%, at least 80%, at least 90%, at least 95%, or 100% relative to a suitable control (for example as compared to prior to treatment with the MCT11 antibody). A decrease in T cell exhaustion can be measured, for example, by a decrease in lactic acid uptake, a decrease in expression of PD-1 or Tim3, an increase in cytokine production (e.g., INF-γ, TNFα, or IL-2), or an increase in cytotoxic activity (e.g., increased tumor specific targeting or killing) or measuring another indicator of T cell effector activity, relative to a suitable control (e.g., measurements from untreated exhausted T cells, or baseline measurements of an exhausted T cell prior to contacting (including administering) or expressing the antibody specific for MCT11). In some embodiments, the method reduces MCT11 activity, for example, by at least 10%, at least 20%, at least 30%, at least 40%, at least 50%, at least 60%, at least 70%, at least 80%, at least 90%, at least 95%, or 100% relative to a suitable control. In some embodiments, the method reduces lactic acid transport or uptake of a T cell (such as an exhausted or terminally exhausted T cell), for example, by at least 10%, at least 20%, at least 30%, at least 40%, at least 50%, at least 60%, at least 70%, at least 80%, at least 90%, at least 95%, or 100% relative to a suitable control (for example as compared to an amount of lactic acid uptake prior to treatment with the MCT11 antibody). In some examples, combinations of these effects are achieved.

In some embodiments, methods disclosed herein increase effector function (e.g., cytokine secretion, cell proliferation, tumor targeting, elimination of cancerous cells) of a T cell, for example, a CD3⁺ or CD8⁺ T cell. In some examples, the methods increase effector function of a T cell in a subject. In some embodiments, the method increases T cell effector function, for example, by at least 10%, at least 20%, at least 30%, at least 40%, at least 50%, at least 60%, at least 70%, at least 80%, at least 90%, at least 95%, at least 100%. at least 150%, at least 200%, at least 250%, at least 300%, at least 400%, at least 500%, or more relative to a suitable control (for example as compared to an amount of effector function prior to treatment with the MCT11 antibody). An increase in effector function of a T cell can be measured, for example, by a decrease in lactic acid uptake, a decrease in expression of PD-1 or Tim3, an increase in cytokine production (e.g., INF-γ, TNFα, or IL-2), an increase in cell proliferation (in vitro or in vivo expansion), or an increase in cytotoxic activity (e.g., increased tumor specific targeting or killing) or other indicator of effector function, relative to a suitable control (e.g., measurements from untreated T cells, or baseline measurements of a T cell prior to contacting (including administering) or expressing the antibody specific for MCT11). In some examples, combinations of these effects are achieved.

In some embodiments, the method includes administering to the subject a MCT11 monoclonal antibody, antigen binding fragment, multispecific antibody, or conjugate disclosed herein and a pharmaceutically acceptable carrier. Doses of the MCT11 antibody, antigen binding fragment, multispecific antibody, or conjugates can vary, but exemplary dosages range between about 0.01 to about 50 mg per kg body weight of the subject, for example, about 0.01 mg/kg to about 20 mg/kg, about 0.5 mg/kg to about 20 mg/kg, about 1 mg/kg to about 20 mg/kg, about 5 mg/kg to about 20 mg/kg, about 10 mg/kg to about 20 mg/kg, about 15 mg/kg to about 20 mg/kg, about 0.01 mg/kg to about 10 mg/kg, about 0.1 mg/kg to about 10 mg/kg, about 0.5 mg/kg to about 10 mg/kg, about 1 mg/kg to about 10 mg/kg, about 5 mg/kg to about 10 mg/kg, about 0.01 mg/kg to about 5 mg/kg, about 0.1 mg/kg to about 5 mg/kg, about 0.5 mg/kg to about 5 mg/kg, about 1 mg/kg to about 5 mg/kg, about 5 mg/kg to about 15 mg/kg, about 5 mg/kg to 20 mg/kg, about 5 mg/kg to 25 mg/kg, about 5 mg/kg to 30 mg/kg, about 5 mg/kg to 35 mg/kg, about 5 mg/kg to 40 mg/kg, about 5 mg/kg to 50 mg/kg, about 10 mg/kg to 20 mg/kg, about 10 mg/kg to 30 mg/kg, about 10 mg/kg to 40 mg/kg, about 10 mg/kg to 50 mg/kg, about 20 mg/kg to 40 mg/kg, or about 20 mg/kg to 50 mg/kg. In some examples, administration is intravenous and includes about 0.5 to about 3 mg/kg of the antibody, antigen binding fragment, multispecific antibody, or conjugate administered once every 2 to 4 weeks. In some examples, the dose is 50 mg, 100 mg, 200 mg, or 500 mg once every 3 weeks.

In some embodiments, the subject is administered DNA or RNA encoding an MCT11 antibody, such as a disclosed MCT11 monoclonal antibody, antigen binding fragment, or multispecific antibody, to provide in vivo antibody production, for example using the cellular machinery of the subject. In specific, non-limiting examples, an effective amount of mRNA encoding an scFV is administered to the subject. Methods for administering exogenous mRNA for in vivo protein expression is disclosed, for example, in Schlake et al. (2019) Molecular Therapy 27(4):773-784, herein incorporated by reference in its entirety.

Any suitable method of nucleic acid administration may be used; non-limiting examples include U.S. Pat. Nos. 5,643,578, 5,593,972 and 5,817,637. U.S. Pat. No. 5,880,103 (herein incorporated by reference in their entirety), which describe several methods of delivery of nucleic acids encoding proteins to a subject.

In some embodiments, the subject is administered an effective amount of the vector that encodes one or more nucleic acid molecules encoding the monoclonal antibody, antigen binding fragment, multispecific antibody, or conjugate disclosed herein. Administration of an effective amount of the vector leads to expression of an effective amount of the monoclonal antibody, antigen binding fragment, multispecific antibody, or conjugate in the subject. In some examples, the nucleic acid or vector is injected, for example, subcutaneously, intramuscularly, intradermally, intraperitoneally, intratumorally, intraprostatic, or intravenously. In specific, non-limiting examples, the nucleic acid or vector is administered by intramuscular injection. In some examples, the dosage for intramuscular injection is about 0.5 μg/kg to about 50 mg/kg, for example, 0.005 mg/kg to about 5 mg/kg (see, e.g., U.S. Pat. No. 5,589,466).

Multiple doses of the MCT11 antibody, antigen binding fragment, multispecific antibody, conjugate, or nucleic acid molecule or vector encoding such molecules, as disclosed herein, may be administered, for example, per day, per week, or per month, according to a dosing schedule determined by a medical practitioner. In some examples, the antibody, antigen binding fragment or multispecific antibody, conjugate, or nucleic acid molecule or vector encoding such molecules, is administered weekly, every two weeks, every three weeks, every four weeks, monthly, or less frequently. In some examples, a treatment is administered every other day for three days. A skilled clinician can select an administration schedule based on the subject, the condition being treated, the previous treatment history, tumor load and type, clinical stage and grade of the disease, overall health, and other factors. The dosage can be administered once, or may be applied periodically until either a desired result is achieved or until side effects warrant discontinuation of therapy. Generally, the dose is sufficient to exhibit a desired response without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

Administration includes local and systemic administration, such as, e.g., by injection subcutaneously, intravenously, intra-arterially, intraperitoneally, intramuscularly, intradermally, or intrathecally. In some embodiments, the antibody, antigen binding fragment, multispecific antibody or nucleic acid molecule encoding such molecules, or a composition including such molecules, is administered by a single subcutaneous, intravenous, intra-arterial, intraperitoneal, intramuscular, intradermal or intrathecal injection once a day. The antibody, antigen binding fragment, multispecific antibody, conjugate, or nucleic acid molecule encoding such molecules, or a composition including such molecules, can also be administered by direct injection at or near the site of disease (e.g., a tumor or cancer location). A further method of administration is by osmotic pump (e.g., an Alzet pump) or mini-pump (e.g., an Alzet mini-osmotic pump), which allows for controlled, continuous and/or slow-release delivery of the antibody, antigen binding fragment, conjugate, or nucleic acid molecule encoding such molecules, or a composition including such molecules, over a pre-determined period. The osmotic pump or mini-pump can be implanted subcutaneously, or near a target site.

In some examples, the methods disclosed herein further include treating the subject with one or more of surgery, radiation, chemotherapy, biologic therapy, or immunotherapy. In some examples, the immunotherapy includes one or more of a checkpoint inhibitor, a T cell agonist antibody, an oncolytic virus (e.g., T-VEC), or an adoptive cell transfer (ACT) immunotherapy.

Exemplary chemotherapeutic agents that can be used in combination with an MCT11 antibody include (but are not limited to) alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine); antimetabolites such as folic acid analogs (such as methotrexate), pyrimidine analogs (such as 5-FU or cytarabine), and purine analogs, such as mercaptopurine or thioguanine; or natural products, for example vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Additional agents include platinum coordination complexes (such as cis-diamine-dichloroplatinum II, also known as cisplatin), substituted ureas (such as hydroxyurea), methyl hydrazine derivatives (such as procarbazine), and adrenocortical suppressants (such as mitotane and aminoglutethimide); hormones and antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include adriamycin, melphalan (Alkeran®) Ara-C (cytarabine), carmustine, busulfan, lomustine, carboplatinum, cisplatinum, cyclophosphamide (Cytoxan®), daunorubicin, dacarbazine, 5-fluorouracil, fludarabine, hydroxyurea, idarubicin, ifosfamide, methotrexate, mithramycin, mitomycin, mitoxantrone, nitrogen mustard, paclitaxel (or other taxanes, such as docetaxel), vinblastine, vincristine, VP-16, while newer drugs include gemcitabine (Gemzar®), trastuzumab (Herceptin®), irinotecan (CPT-11), leustatin, navelbine, rituximab (Rituxan®) imatinib (STI-571), Topotecan (Hycamtin®), capecitabine, ibritumomab (Zevalin®), and calcitriol. A skilled clinician can select appropriate additional therapies (from those listed here or other current therapies) for the subject, depending on factors such as the subject, the cancer being treated, treatment history, and other factors.

In some examples, the method further includes treating the subject with an additional therapeutic, such as a monoclonal antibody (e.g., anti-CTLA-4, anti-PD1, or anti-PDL1), a T cell agonist antibody (e.g., urelumab and utomilumab), an oncolytic virus, an adoptive cell transfer (ACT) therapy, or any combination of two or more thereof. In some examples, the additional therapeutic is a cell cycle or checkpoint inhibitor. In some examples, the checkpoint inhibitor targets PD-1, PD-L1, CTLA-4, CDK4, and/or CDK6. Exemplary inhibitors include ipilimumab, nivolumab, pembrolizumab, atezolizumab, avelumab, durvalumab, cemiplimab, palbociclib, ribociclib, and abemaciclib. In some examples, the subject is administered an ACT therapy, for example, a chimeric antigen receptor (CAR)-expressing T cell, engineered TCR T cell, or a tumor-infiltrating lymphocyte (TIL).

The additional therapeutic may be administered substantially simultaneously with the disclosed composition. In some examples, the additional therapeutic is administered prior to administering the composition, for example, at least 1 day, at least 2 days, at least 3 days, at least 4 days, at least 5 days, at least 6 days, at least 7 days, at least 8 days, at least 9 days, at least 12 days, at least 14 days, at least three weeks, at least four weeks, at least one month, or more prior. Multiple doses of the additional therapeutic can be administered to a subject, for example, administered twice daily, once daily, every other day, twice per week, weekly, every other week, every three weeks, monthly, or less frequently. A skilled clinician can select an administration schedule based on the subject, the condition being treated, the previous treatment history, tumor load and type, clinical stage and grade of the disease and overall health of the subject, and other factors.

Also provided are compositions or kits that can be used with the disclosed methods. In some examples, the composition or kit includes one or more MCT11 antibody, antigen binding fragment, multispecific antibody, conjugate, or nucleic acid molecule or vector encoding such molecules, for example, in a pharmaceutically acceptable carrier. The kit can include additional reagents, such as one or more additional antibodies (e.g., anti-CD3, anti-CD8, anti-CD28, anti-CD44, anti-PD1, anti-TIM3), transfection reagents, vectors, culture medium, antibiotics, or cytokines (e.g., IL-2, IL-15, and IL-7). The kit can include cells, for example, cells for protein expression. In some examples, the reagents are present in separate containers.

### EXAMPLES

### Example 1

### Materials and Methods

This example provides the materials and methods used to generate the data discussed in the Examples below.

### RNA-seq

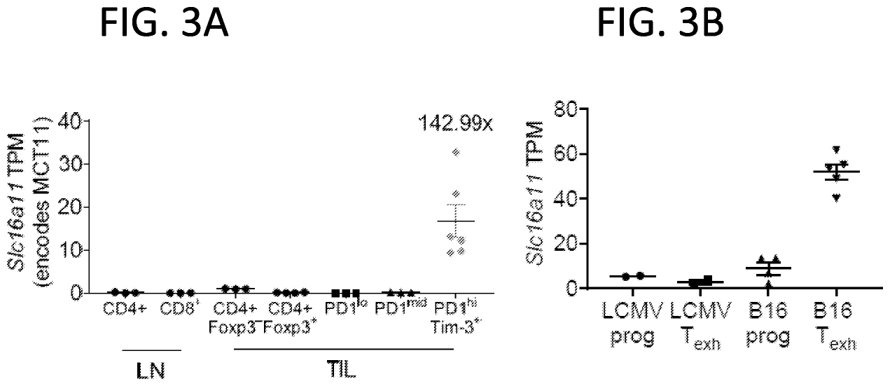

C57/BL6 mice were implanted with B16 melanoma. When tumors reached 7 mm in any direction, LN and tumor were harvested and processed, and CD8⁺ T cells were sorted on the basis of CD44, PD-1, and Tim-3 expression from the lymph node (LN) and tumor infiltrating lymphocytes (TIL). RNA-seq was performed on 1,000 cells isolated from the following compartments: LN CD44^{hi}, TIL PD-1^{lo}, TIL PD-1^{mid}, TIL PD-1^{hi}, and TIL PD-1^{hi}Tim3⁺. CD4⁺ T cells from LN and TIL were sequenced from a separate experiment. RNA was prepared from cell lysates of 1000 cells using the Clontech SMARTer® kit, and sequenced on an Illumina NextSEQ®. TPMs were calculated after aligning to the mouse genome (mm9 assembly). Transcripts per million (TPM) plots of Slc16a11 (encoding MCT11) are shown (FIG. 3A).

### Lactic Acid Uptake

C57/BL6 mice were implanted with B16 melanoma as above, and TIL preparations were made. TIL was loaded with pHrodo® Red, a pH sensitive dye, and incubated in Hank's Balanced Salt Solution (HBSS). Lactic acid was pulsed for 30 mins and pH change was measured by flow cytometry, as described in Watson et al. (2021) Nature, 591: 645-651, herein incorporated by reference in its entirety. In experiments that tested antibody inhibition of MCT11, preparations were incubated with 10 μg/mL of polyclonal or monoclonal anti-MCT11 (see, Example 3) antibodies prior to pulsing with LA.

### In Vivo Experiments

### B16 Melanoma

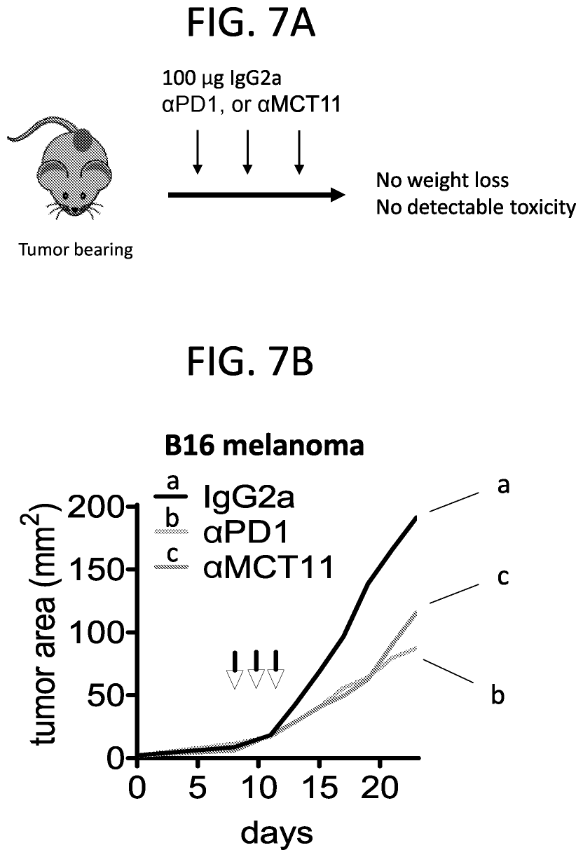

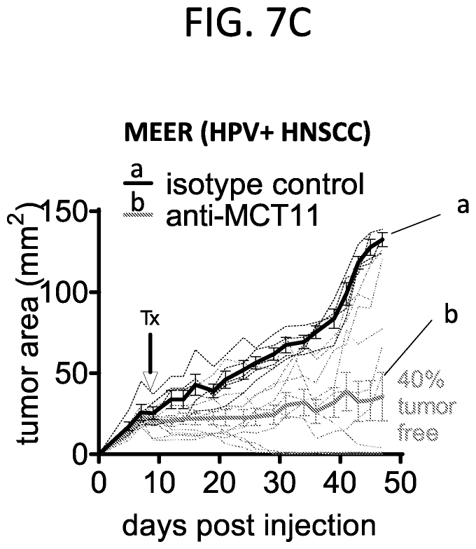

Mice received subcutaneous injection of 1×10⁵ tumor cells. Mice bearing 3 mm diameter B16 melanoma tumors were treated every other day for 3 treatments with 100 μg/mouse MCT11 mAb (see, Example 3), PD-1 mAb, or isotype control, via the intraperitoneal route (see, FIG. 7A). Tumor growth inhibition was followed.

### MEER (HPV Positive Head and Neck Squamous Cell Carcinoma (HNSCC)

Mice received subcutaneous injection of 1×10⁵ tumor cells. Mice bearing 3 mm diameter tumors were treated every other day for 3 treatments with 100 μg/mouse MCT11 mAb (see, Example 3) or isotype control via the intraperitoneal route. Tumor growth inhibition was followed.

### Fc Mutant Antibody (LALAPG)

Mice received a subcutaneous injection of 1×10⁵ MEER tumor cells. Mice bearing 3 mm diameter tumors were treated with 100 μg/mouse isotype control, MCT11 mAb (see, Example 3), or the mutant MCT11 mAb, via the intraperitoneal route. Tumor growth inhibition was followed.

Mice that cleared tumors in response to MCT11 blockade were inoculated again with MEER tumor cells at least 30 days after clearance at 1×10⁵ MEER cells/mouse in the absence of any treatment. Tumor growth inhibition was followed.

### Example 2

### Discovery of MCT11 on Exhausted T Cells

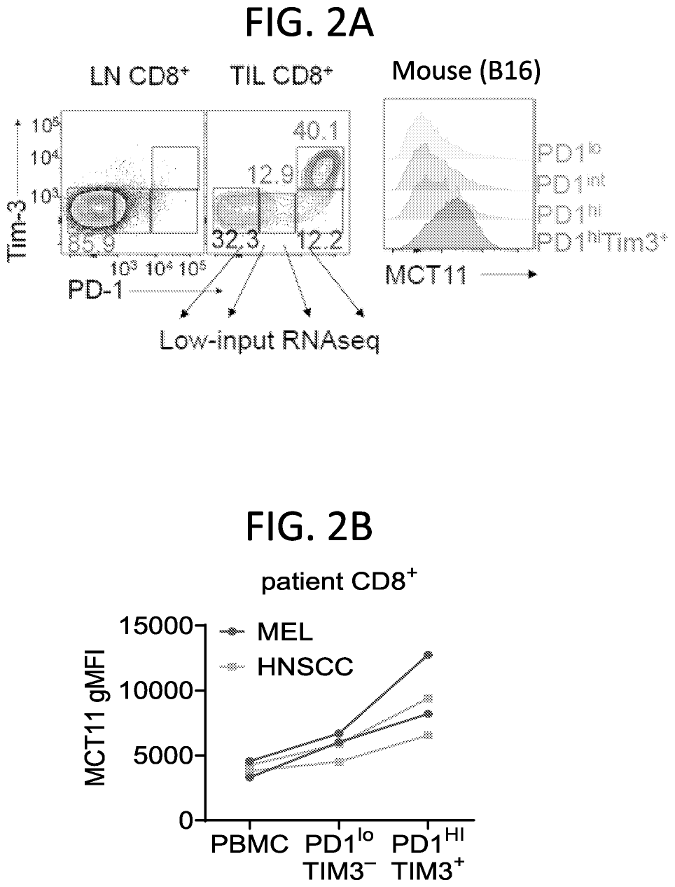

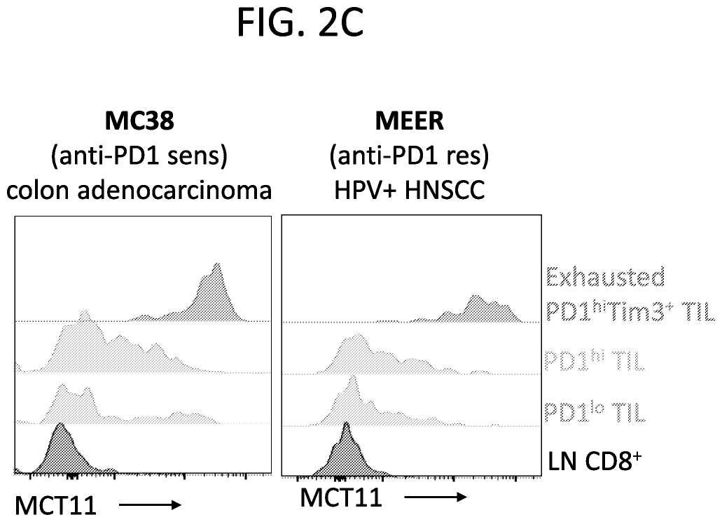

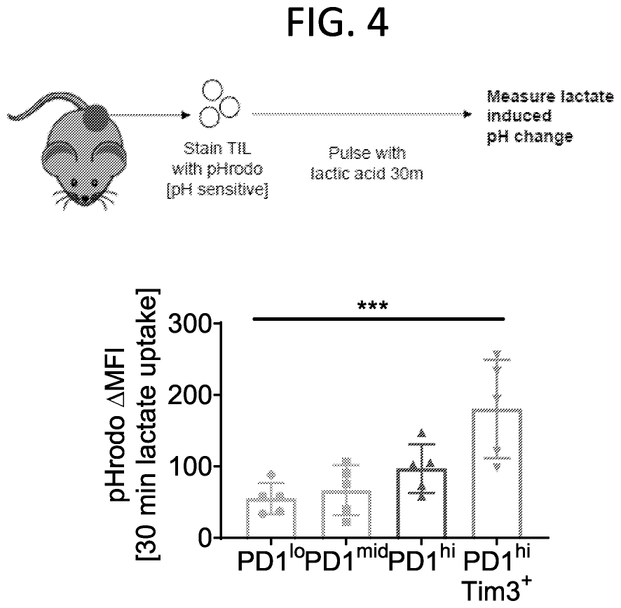

Using RNA-seq and metabolic profiling, terminally exhausted T cells (dysfunctional T cells common in cancer environments) were found to highly express a novel nutrient transporter called MCT11 (encoded by Slc16a11) (FIGS. 3A and 3B). MCT11 likely transports monocarboxylates, short chain carbon sources such as lactic acid, pyruvate, and short-chain fatty acids. MCT11 upregulation in exhausted T cells in human and mice was confirmed by flow cytometry and RNA-Seq (see, FIGS. 2A-2C and FIGS. 3A-3B). Further, it was confirmed that terminally exhausted T cells specifically take up monocarboxylates, such as lactic acid (FIG. 4). However, MCT11 is not expressed on the surface of exhausted T cells induced by chronic viral infection. These findings indicate that MCT11 may be important in providing nutrient flux to terminally exhausted T cells.

### Example 3

### Monoclonal MCT11 Antibody Generation

Mice were immunized against MCT11 and monoclonal antibodies were generated from fusion with myeloma cells as standard practice. Single cell cloning and selection of well producing clones was followed by screening for binding against the immunizing peptide (SEQ ID NO: 11). The clone was selected based on surface binding to MCT11-overexpressing cell lines but not those deficient in MCT11. Sequence information for the MCT11 mAb is provided in Table 1.

### Example 4

### MCT11 Blockade of Lactic Acid Uptake

Non-exhausted tumor-infiltrating T cells (TILs not expressing MCT11), and LN-derived T cells, do not significantly take up lactic acid. In contrast, terminally exhausted tumor-infiltrating T cells (TILs expressing high levels of PD-1 and Tim-3) actively take up lactic acid (FIG. 4). Whether blockade of MCT11 could inhibit lactic acid uptake in terminally exhausted T cells was investigated.

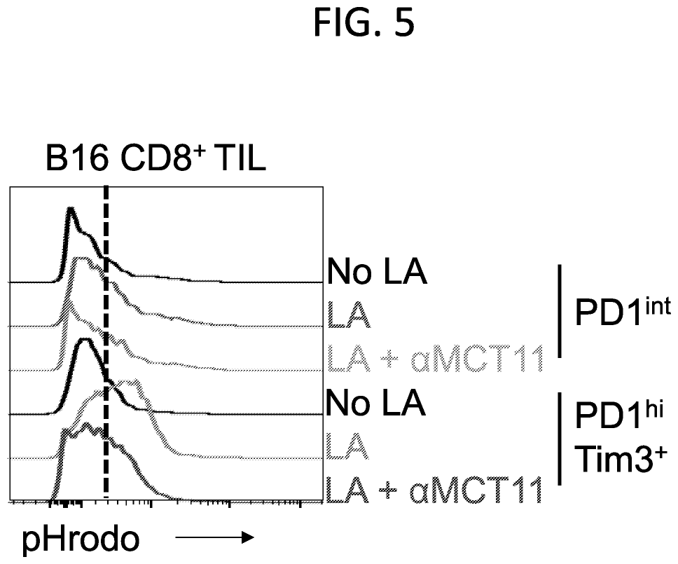

Mice were implanted with B16 melanoma. After tumors reached 5 mm diameter, lymph node (LN) and tumor infiltrating lymphocytes (TIL) preparations were loaded with pH indicator dye pHrodo® red, stained on the cell surface for CD8, PD-1, Tim-3. Prior to pulsing with LA, preparations were incubated with 10 μg/mL of polyclonal anti-MCT11 antibodies (Abcam, ab230845). Samples were then pulsed with 5 mM lactic acid (LA) for 30 minutes. Lactic acid uptake was blocked by treating exhausted T cells with the polyclonal antibody (FIG. 5).

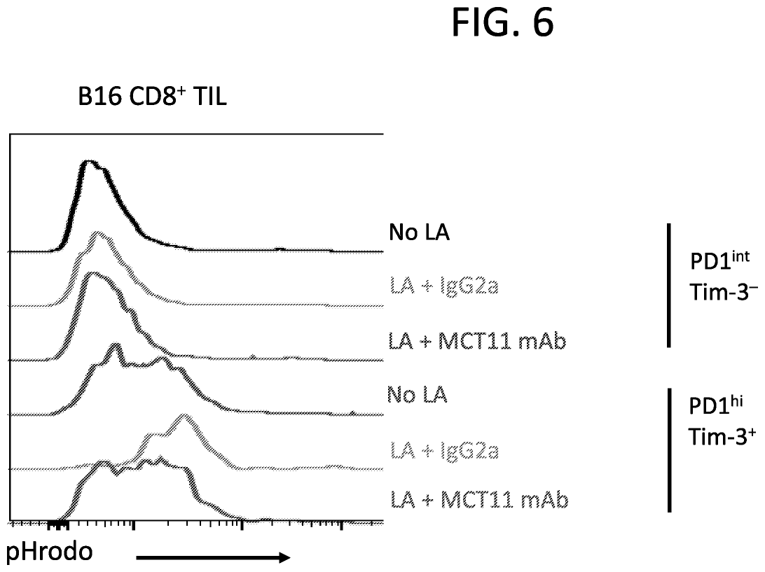

The lactic acid uptake assay was repeated using the purified monoclonal antibody (mouse IgG2a(k) isotype) specific to MCT11 (described in Example 3), and showed again that incubation with 10 μg/mL of anti-MCT11 could prevent lactic acid uptake in terminally exhausted T cells (FIG. 6). This demonstrates that MCT11 supports uptake of lactic acid, and an antibody-mediated approach can be used to prevent uptake of this toxic metabolite.

### Example 5

### MCT11 Blockade In Vitro

To test the functional significance of MCT11 blockade, TIL preparations are restimulated with PMA/ionomycin in the presence of 5 mM lactic acid for 5 hours in vitro with or without a MCT11-specific antibody. The ability of the TILs to produce cytokines and granzyme B is measured, as well as lactic acid uptake.

### Example 6

### Functional Modulation of the Anti-Tumor Response In Vivo

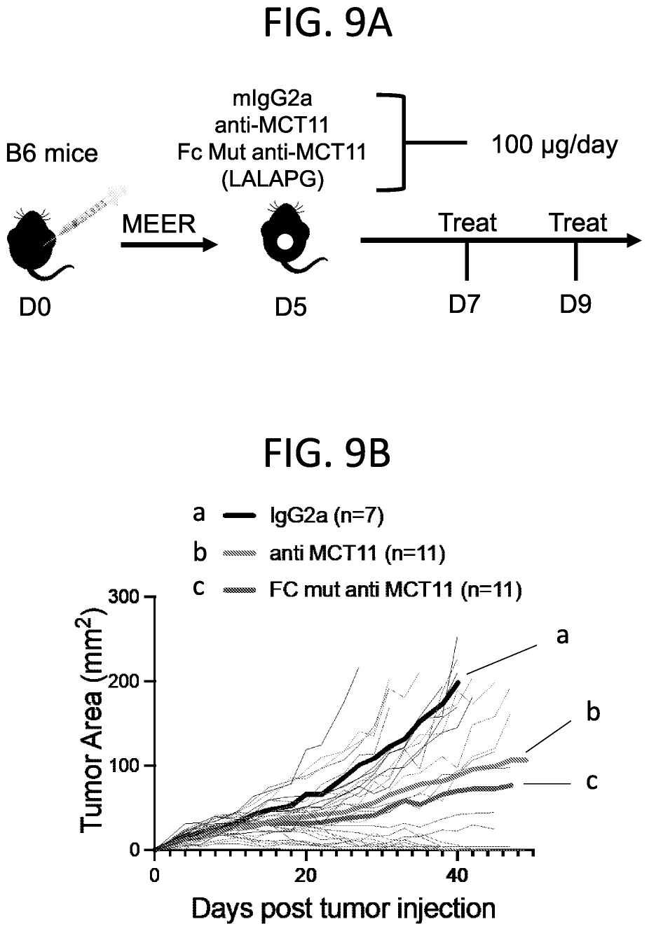

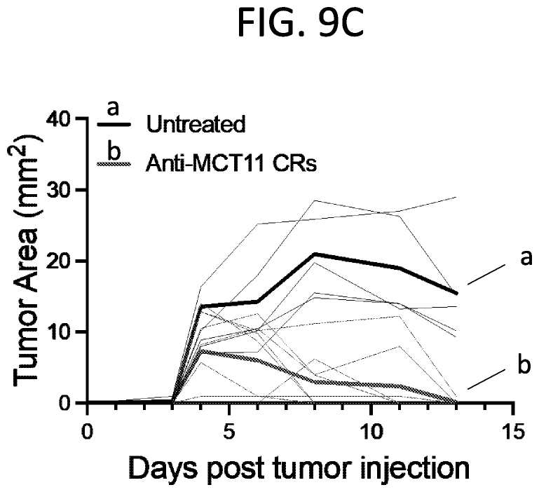

It was investigated whether mAb-mediated blockade of MCT11 could functionally modulate the anti-tumor immune response in vivo by treating a mouse model bearing B16 melanoma tumors. MCT11 blockade by the MCT11 mAb (see, Example 3) demonstrated similar functional effects of PD-1 blockade in the aggressive B16 model of melanoma, and significantly reduced tumor growth (FIG. 7B). Similarly, mAb-mediated blockade of MCT11 was also tested in a mouse model bearing MEER tumors (HPV-positive HNSCC). MCT11 blockade by the MCT11 mAb significantly reduced tumor growth in the MEER model (FIG. 7C). Mice that cleared tumors following α-MCT11 mAb treatment also showed immunologic memory when re-inoculated with MEER tumor cells (FIG. 9C). Notably, mice were healthy and experienced no weight loss, indicating MCT11 blockade was not toxic. This is consistent with the fact that MCT11 germline knock-out mice have no overt phenotype. Thus, MCT11 blockade can be used to block lactic acid uptake on terminally exhausted T cells and rescue cellular function.

### Example 7

### MCT11 Blockade Mechanisms

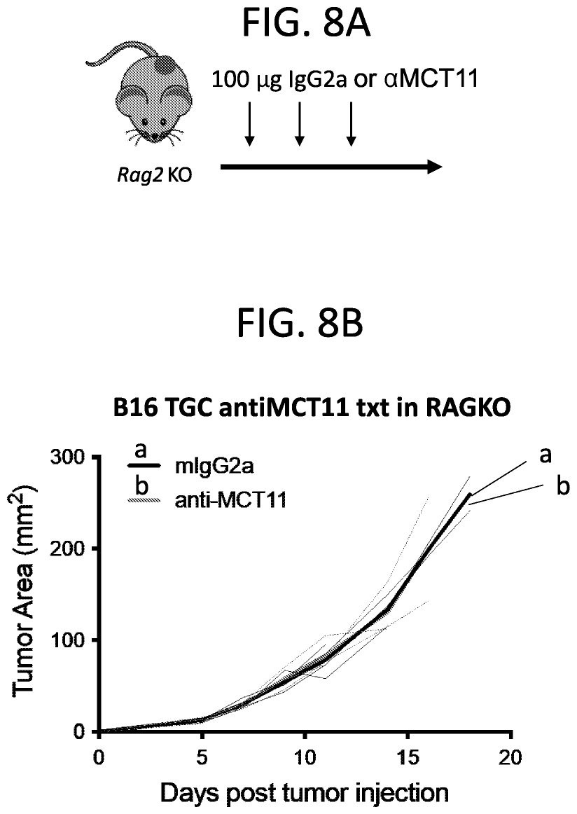

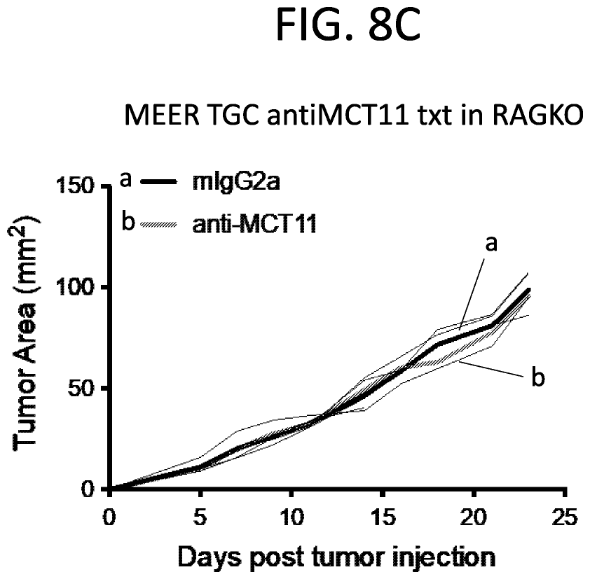

To determine whether MCT11 blockade acts via adaptive immunity, experiments similar to those described in Example 6 were conducted in RAG1-deficient mice (RAGKO), which lack B or T cells. Little difference was observed between the isotype control (IgG2a) and the α-MCT11 mAb (see, Example 3) treatment, indicating that α-MCT11 acts via the immune system (see, FIGS. 8A-8C). In addition, an Fc mutant (LALAPG) of the α-MCT11 mAb was created to determine whether the α-MCT11 mAb functions as a blocking antibody, or if it causes depletion of MCT11-expressing cells. B6 mice inoculated with MEER tumor cells were treated with an isotype control, α-MCT11 mAb, or the mutant α-MCT11 mAb (Fc mut anti MCT11) (FIGS. 9A and 9B). The results show that the α-MCT11 mAb functions as a blocking antibody rather than by depleting MCT11-expressing cells.

### Example 8

### Administering Monoclonal α-MCT11 to Treat Cancer

In this example, an effective amount (e.g., 200 mg intravenously once every three weeks) of a monoclonal antibody that specifically binds MCT11 (e.g., a commercially available MCT11 antibody, or the MCT11 mAb of Example 3) is administered intravenously to a patient in need of cancer treatment. The antibody can be administered alone, or in combination with other immunotherapeutic regimens, for example, checkpoint blockage antibodies (PD-1, CTLA4, LAG3), T cell agonist antibodies (41BB, OX40, GITR), oncolytic viruses (T-VEC, etc.), or ACT (CAR-T, TCR-T, TIL) therapy.

In a specific example, the MCT11-specific antibody is administered before, after, or substantially at the same time as an immunotherapy, such as abemaciclib, atezolizumab, avelumab, axicabtagene ciloleucel, blinotumumabl, cemipilimab, durvalumab, ieramilimab, ipilimumab, nivolumab, palbociclib, pembrolizmab, pidilizumab, relatlimab, ribociclib, urelemab, utolimumab, adoptive cell transfer (ACT) therapy (e.g., chimeric antigen receptor (CAR) (e.g., tisagenlecleucel)), or engineered TCR or tumor-infiltrating lymphocyte (TIL)), or oncolytic viruses (e.g., talimogene laherparepvec (T-VEC)). In some examples, the MCT11-specific antibody is administered substantially at the same time as the immunotherapy. One or more signs or symptoms of cancer (or a tumor) are periodically measured, for example, tumor size, tumor load, tumor density, clinical grade, presence of metastasis, number of metastasis, morbidity, mortality, or other measurements (qualitative or quantitative). Measurements can be compared, for example, to measurements obtained prior to administering the MCT11-specific antibody to the subject, or can be compared to a control group, for example, subjects not administered the MCT11-specific antibody. In this example, administering the MCT11-specific antibody improves one or more signs or symptoms of cancer (or a tumor).

### Example 9

### Use of α-MCT11 to Reduce Exhausted T Cells

In this example, an effective amount of an MCT11 antibody, such as a monoclonal antibody (e.g., the MCT11 mAb of Example 3 or a commercially available MCT11 antibody), is administered to remove exhausted T cells from a sample. In some embodiments, the method includes contacting the sample with an effective amount of an antibody specific for MCT11, and removing cells bound to the antibody from the sample, thereby generating a sample depleted of exhausted T cells.

The sample can be a sample of cells, for example, a PBMC sample or a population of T cells. The method can include obtaining a PBMC sample, for example, from a subject, such as a subject with cancer. The population of T cells can include a population of cells for ACT therapy, such as CAR-T, TCR, and TIL cells for use in ACT therapy. The sample of cells can be cultured, for example to expand the cells (such as expanding T cells), before or after contacting with the sample with the antibody specific to MCT11 (or both before and after).

In a specific, non-limiting example, the sample is a PBMC sample. The PBMCs can be cultured ex vivo, for example to expand the cells (such as T cells). Exhausted T cells (such as terminally exhausted T cells) can be removed from the PBMC population (either PBMCs directly obtained from the subject, or PBMCs subsequently expanded ex vivo), by contacting the PBMCs with an MCT11 antibody, such as a monoclonal antibody. Cells in the PBMC population that bind to the MCT11 antibody (which are the exhausted T cells) can be separated from the other cells in the PBMC population, thus enriching the PBMC population for T cells that are not exhausted, or are not terminally exhausted. In some examples, such methods remove at least 20% of the exhausted T cells in the PBMC population (such as terminally exhausted T cells), for example, at least 30%, at least 40%, at least 50%, at least 60%, at least 70%, at least 75%, at least 80%, at least 85%, at least 90%, at least 95%, at least 99%, or at least 99.9% of the exhausted T cells (such as terminally exhausted T cells). In some examples, such methods produce a population of PBMC cells that is substantially free of exhausted T cells (such as terminally exhausted T cells).

Exhausted T cells (including terminally exhausted T cells) can be removed from the sample, for example, by flow cytometry, magnetic separation, or panning. In one example, the PBMC sample or population of T cells are incubated with an MCT11 antibody, and with an appropriately labeled secondary antibody (such as one including a fluorophore), allowing the labeled cells to be separated from the non-labeled cells (e.g., using flow cytometry). In some examples, the MCT11 antibody is directly labeled with a fluorophore instead of using a secondary antibody. In one example, magnetic separation is used (for example by using a paramagnetic particles coated with the MCT11 antibody, which is incubated with the PBMCs, and the cells that are not attached to the particles separated, for example by using centrifugation and/or washing, wherein the supernatant is collected). In one example, panning is used (for example by using a solid support coated with the MCT11 antibody, which is incubated with the PBMCs, and the cells that are not attached to the support collected, for example by washing).

In some examples, the method results in a sample depleted of exhausted T cells (such as terminally exhausted T cells) that is used in an anti-cancer immunotherapy, such as the methods provided herein.

In another example, an effective amount of an MCT11 antibody, such as a monoclonal antibody (e.g., the MCT11 mAb of Example 3 or a commercially available MCT11 antibody), is administered to deplete exhausted T cells from a subject. In some embodiments, the method includes administering to the subject an effective amount of an antibody specific for MCT11, thereby depleting exhausted T cells in the subject. In some examples, a subject having cancer, or receiving an immunotherapy, is selected for treatment.

In view of the many possible embodiments to which the principles of the disclosed invention may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A monoclonal antibody that specifically binds monocarboxylate transporter 11 (MCT11), comprising a variable heavy (V_{H}) domain and a variable light (V_{L}) domain, wherein:
the V_{H} domain comprises the heavy chain complementarity determining region (CDR) 1, a CDR2, and a CDR3 of SEQ ID NO: 1, and
the V_{L} domain comprises the light chain complementarity determining region (CDR)1, a CDR2, and a CDR3 of SEQ ID NO: 5.

2. (canceled)

3. The monoclonal antibody of claim 1, wherein:
the heavy chain CDR1, CDR2, and CDR3 comprise the amino acids sequences set forth as SEQ ID NOs: 2, 3, and 4, respectively, and
the light chain CDR1, CDR2, and CDR3 comprise the amino acids sequences set forth as SEQ ID NOs: 6, 7, and 8, respectively.

4. The monoclonal antibody of claim 1, wherein:
the amino acid sequence of the V_{H} domain is at least 90% identical to SEQ ID NO: 1 and comprises the heavy chain CDR1, CDR2 and CDR3 sequences of SEQ ID NO: 1, and
the amino acid sequence of the V_{L} domain is at least 90% identical to SEQ ID NO: 5 and comprises the light chain CDR1, CDR2 and CDR3 sequences of SEQ ID NO: 5.

5. The monoclonal antibody of claim 1, wherein:
the V_{H} domain comprises or consists of SEQ ID NO: 1, and
the V_{L} domain comprises or consists of SEQ ID NO: 5.

6. The monoclonal antibody of claim 1, wherein the antibody is an antigen binding fragment selected from: an Fab fragment, an Fab′ fragment, an F(ab)′₂ fragment, an Fv, a single chain variable fragment (scFV), a dimer of a single chain antibody (scFV₂), and a disulfide stabilized variable fragment (dsFV).

7. The monoclonal antibody of claim 1, wherein the monoclonal antibody is a mouse antibody, a humanized antibody, a human antibody, or a chimeric antibody.

8-10. (canceled)

11. The monoclonal antibody of claim 1, comprising a constant region, optionally wherein the constant region comprises at least one modification to increase half-life, stability and/or function of the monoclonal antibody.

12. (canceled)

13. An antibody conjugate, comprising the monoclonal antibody of claim 1 linked to an effector molecule, therapeutic, or a detectable marker.

14. (canceled)

15. A multispecific antibody comprising the monoclonal antibody of claim 1 and at least one antibody that specifically binds an additional antigen.

16. (canceled)

17. An isolated nucleic acid molecule encoding the monoclonal antibody of claim 1.

18-19. (canceled)

20. A vector comprising the nucleic acid molecule of claim 17.

21. A host cell comprising the nucleic acid molecule of claim 17 or vector encoding the nucleic acid molecule.

22. A composition comprising the monoclonal antibody of claim 1 or a nucleic acid encoding the antibody and a pharmaceutically acceptable carrier.

23. A method for reducing T cell exhaustion, reducing lactic acid uptake by an exhausted T cell, increasing effector function of a T cell, or combinations thereof, comprising:
(a) contacting the exhausted T cell or T cell with an effective amount of an antibody specific for monocarboxylate transporter 11 (MCT11); or
(b) expressing a nucleic acid molecule or a vector encoding the antibody specific for MCT11 in the exhausted T cell or T cell;
thereby reducing T cell exhaustion, reducing lactic acid uptake by an exhausted T cell, increasing effector function of a T cell, or combinations thereof.

24. The method of claim 23,
wherein the antibody specific for MCT11 comprises a variable heavy (V_{H}) domain and a variable light (V_{L}) domain, and wherein:
the V_{H} domain comprises the heavy chain complementarity determining region (CDR) 1, a CDR2, and a CDR3 of SEQ ID NO: 1, and
the V_{L} domain comprises the light chain complementarity determining region (CDR)1, a CDR2, and a CDR3 of SEQ ID NO: 5.

25-28. (canceled)

29. A method for treating cancer or a tumor, or increasing an immune response in a subject, comprising administering to the subject a therapeutically effective amount of an antibody specific for monocarboxylate transporter 11 (MCT11),
thereby treating the cancer or tumor, or increasing the immune response.

30. The method of claim 29, wherein the subject has cancer or is receiving an immunotherapy.

31. (canceled)

32. The method of claim 30, wherein the immunotherapy comprises tumor-infiltrating lymphocyte (TIL) therapy, chimeric antigen receptor T cell (CAR-T) therapy, or engineered T cell receptor (TCR) therapy.

33-35. (canceled)

36. A method of removing exhausted T cells from a sample, comprising:
(1) contacting the sample with an effective amount the antibody of claim 1, and
(2) removing cells bound to the antibody, generating a sample depleted of exhausted T cells.

37-43. (canceled)

44. A method of increasing an immune response in a subject being treated with an antibody specific for monocarboxylate transporter 11 (MCT11), comprising administering an immunotherapy to the subject, optionally wherein the subject has cancer.

45-55. (canceled)


SEQUENCE LISTING (13 sequences)

SEQ ID NO: 1 - protein, 452 aa, synthetic construct
QVTLKESGPGILQPSQTLSLTCSFSGFSLSTSGLGVGWIRQPSGKGLEWLSHIWWNDVKRYNPALESRLTISKDTSSSQVFLKIASVDTSDTATYYCARIIRSSLITGAVDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 2 - protein, 9 aa, synthetic construct
GFSLSTSGL

SEQ ID NO: 3 - protein, 5 aa, synthetic construct
WWNDV

SEQ ID NO: 4 - protein, 13 aa, synthetic construct
IIRSSLITGAVDY

SEQ ID NO: 5 - protein, 214 aa, synthetic construct
DIQMTQTTSSLSASLGDRVTISCSASQGIYNYLNWYRQKPDGTVELLIYYTSSLHSGVPSRFSGSGSGTAYSLTISNLEPEDFATYYCQQYRKLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - protein, 11 aa, synthetic construct
SASQGIYNYLN

SEQ ID NO: 7 - protein, 7 aa, synthetic construct
YTSSLHS

SEQ ID NO: 8 - protein, 9 aa, synthetic construct
QQYRKLPWT

SEQ ID NO: 9 - protein, 471 aa, Homo sapiens
MPAPQRKHRRGGFSHRCFPTPQTAMTPQPAGPPDGGWGWVVAAAAFAINGLSYGLLRSLGLAFPDLAEHFDRSAQDTAWISALALAVQQAASPVGSALSTRWGARPVVMVGGVLASLGFVFSAFASDLLHLYLGLGLLAGFGWALVFAPALGTLSRYFSRRRVLAVGLALTGNGASSLLLAPALQLLLDTFGWRGALLLLGAITLHLTPCGALLLPLVLPGDPPAPPRSPLAALGLSLFTRRAFSIFALGTALVGGGYFVPYVHLAPHALDRGLGGYGAALVVAVAAMGDAGARLVCGWLADQGWVPLPRLLAVFGALTGLGLWVVGLVPVVGGEESWGGPLLAAAVAYGLSAGSYAPLVFGVLPGLVGVGGVVQATGLVMMLMSLGGLLGPPLSGFLRDETGDFTASFLLSGSLILSGSFIYIGLPRALPSCGPASPPATPPPETGELLPAPQAVLLSPGGPGSTLDTTC

SEQ ID NO: 10 - DNA, 1726 bp, synthetic construct
  source 1..1726: mol_type = other DNA
catttctcgccgctcccccctcccccgggcctgggggttgtgtgtgtacataattcaatccccgtgggactggcgtctggcctcacgcgcggcttgccggattggctgtttcagctcgcccccgccctccgtacaccccgcgtcccaggtggctcaggcccctgtggtgatctctgtttaccgagagagcccgtccaagttgggctccatcgctgccctcgctccccttcggggcccccgcccgcctgggaagcagagagaaagccgggcccagcccttcctcacccttcccctccccgcaccgcccggagaggtcggacggcgatgaccccccagcccgccggacccccggatgggggctggggctgggtggtggcggccgcagccttcgcgataaacgggctgtcctacgggctgctgcgctcgctgggccttgccttccctgaccttgccgagcactttgaccgaagcgcccaggacactgcgtggatcagcgccctggccctggccgtgcagcaggcagccagccccgtgggcagcgccctgagcacgcgctggggggcccgccccgtggtgatggttgggggcgtcctcgcctcgctgggcttcgtcttctcggctttcgccagcgatctgctgcatctctacctcggcctgggcctcctcgctggctttggttgggccctggtgttcgcccccgccctaggcaccctctcgcgttacttctcccgccgtcgagtcttggcggtggggctggcgctcaccggcaacggggcctcctcgctgctcctggcgcccgccttgcagcttcttctcgatactttcggctggcggggcgctctgctcctcctcggcgcgatcaccctccacctcaccccctgtggcgccctgctgctacccctggtccttcctggagaccccccagccccaccgcgtagtcccctagctgccctcggcctgagtctgttcacacgccgggccttctcaatctttgctctaggcacagccctggttgggggcgggtacttcgttccttacgtgcacttggctccccacgctttagaccggggcctggggggatacggagcagcgctggtggtggccgtggctgcgatgggggatgcgggcgcccggctggtctgcgggtggctggcagaccaaggctgggtgcccctcccgcggctgctggccgtattcggggctctgactgggctggggctgtgggtggtggggctggtgcccgtggtgggcggcgaagagagctgggggggtcccctgctggccgcggctgtggcctatgggctgagcgcggggagttacgccccgctggttttcggtgtactccccgggctggtgggcgtcggaggtgtggtgcaggccacagggctggtgatgatgctgatgagcctcggggggctcctgggccctcccctgtcaggcttcctaagggatgagacaggagacttcaccgcctctttcctcctgtctggttctttgatcctctccggcagcttcatctacatagggttgcccagggcgctgccctcctgtggtccagcctcccctccagccacgcctcccccagagacgggggagctgcttcccgctccccaggcagtcttgctgtccccaggaggccctggctccactctggacaccacttgttgattattttcttgtttgagcccctcccccaataaagaatttttatcgggttttcctgaaa

SEQ ID NO: 11 - protein, 47 aa, synthetic construct
VHLAPHALDRGLGGYGAALVVAVAAMGDAGARLVCGWLADQGWVPLP

SEQ ID NO: 12 - protein, 447 aa, Mus musculus
MTPKPAGPPDGGWGWVVAAAAFAVNGLSYGLLRSLGLALPDLAEHFERSAQDTAWVSALALAVQQAASPVGSALSTRWGARPVVMVGGVLTSLGLVFSAFARSLLHLYLGLGLLAGSGWALVFAPALGTLSRYFSRRRVLAVGLALTGNGASSLLLAPALQFLLDTFGWRGALLLLGAVTLHLTPCGALLRPLALSGDPLAPPRTPLAALGLGLFKRRAFSVFALGTALIGGGYFVPYVHLGPHALDQGMGGYGAALVVAVAAVGDACARLASGWLADQGWVPLPRLLVVFGSLTGLGVLAMGLVPTVGTEEGWGAPLLAAAGAYGLSAGSYAPLVFGVLPGLVGIGGVVQATGLVMMLMSLGGLLGPPLSGFLRDKTGDFSASFLVCSSFILSGSFIYMGLPRALPSCRPASPPATPPPERGELLPVPQVSLLSAGGTGSIRDTTC

SEQ ID NO: 13 - DNA, 1773 bp, synthetic construct
  source 1..1773: mol_type = other DNA
gaggcggggcgcggccaccaggtacgctcactcctcgcggttccccaaccagggctggggctcgtgtgtggataattcaatcctcgtgggactcggcgtcaggccttagccaggcctgggtggattggctgtctctctttcccccgccctcggtattcacccccacccccaccccgtgtcccaggtggcttgggcccctgtggtgatctctgtttactggagagagcggtccaagttgggctccatctctgtgctggctagctgctccgcagaaccctttctgcggaaagcgcggagaaaatccggccgaacccaccttcgccctttccctacccccaacgccgggagaggtcggcagacggcgatgacccccaagccggccggacccccggacgggggctggggctgggtggtggcggccgcagcattcgccgtgaacgggctctcctacgggctcttacgctccctgggccttgccctccccgacctcgcggagcactttgaacgaagcgcccaggacactgcgtgggtcagcgccctggccttggccgtgcagcaggcagccagcccagtgggcagcgccctgagcactcgctggggggcacgccccgtggtgatggttgggggagtcctaacctcgcttggcttggtcttctcggctttcgcccgaagcctcctgcacctctacctcggcctgggcctcctcgctggctctggctgggccctggtgtttgccccagccctgggtaccctctctcggtacttctcccgccgtcgggtcttggcggtagggttggcgctcaccggtaatggggcatcctcgctgctcctggcacctgccttgcagttcctccttgatactttcggctggcggggtgccttgctcctccttggcgctgtcacccttcacctcacaccctgtggcgccttgctaagacctttagctctctctggtgacccgctggccccacctcgcacccccctagctgcccttggcctaggtctgttcaagcgccgggccttttcagtctttgctttgggcaccgccttgatcgggggcggatacttcgtcccctacgttcatttgggtccgcatgctttagatcaaggcatgggtggttatggggcagcgttagtggtggctgtcgctgcagtgggagatgcctgtgcccgattggccagcggatggctggcagaccagggctgggtgccccttccgaggcttctggtggtgtttgggtctctgactgggttaggggtactagcaatgggactagtgcccactgtggggacagaggagggttggggggctcctctgctggccgctgctggggcctatgggctgagcgctgggagttatgccccactggttttcggtgtgctcccggggctggtgggcattggaggtgtggtgcaggccacagggctggtgatgatgctgatgagcctcgggggactcctgggccctcctctgtcaggcttcctaagggataagacaggagacttcagtgcctctttcctggtgtgcagctctttcatcctctctggcagtttcatctacatggggctgcccagagccctcccctcctgccgtccagcctcacctccagcaacccctccaccagagagaggggagctgctcccagttccacaagtctccctgctttccgcagggggtactggctccatccgggataccacttgttgatcattttcttggttgacctccttccctaataaagaatttttatcttacaaaaaaaaaaaaaaaaa